(19) United States
(12) Patent Application Publication
(10) Pub. No.: US 2026/0207503 A1
(43) Pub. Date: Jul. 23, 2026

(54) LIPID NANOPARTICLES FOR NUCLEIC ACID DELIVERY

(71) Applicant: ROQUETTE FRERES, Lestrem (FR)

(72) Inventors: Tao PENG, Singapore (SG); Yuan Hao Lucas GOH, Singapore (SG); Shih Chieh CHANG, Singapore (SG)

(21) Appl. No.: 19/137,511

(22) PCT Filed: Dec. 14, 2023
(86) PCT No.: PCT/EP2023/025524

§ 371 Date: Jun. 10, 2025

(30) Foreign Application Priority Data
Dec. 15, 2022 (EP) 22306900.6

(51) Int. Cl. A61K 9/1272; A61K 9/1277; A61K 47/28; A61K 47/40; A61K 48/00

(52) U.S. Cl. CPC A61K 9/1272; A61K 9/1277; A61K 47/28; A61K 47/40; A61K 48/0033

(57) ABSTRACT

The invention relates to new Lipid nanoparticles (LNPs) which can be used for nucleic acid delivery, wherein said LNPs comprise a nucleic acid and a nonionic or cationic cyclodextrin compound. The invention also relates to a process for making the same, and to the use thereof in the pharmaceutical field.

### TECHNICAL FIELD

The invention pertains to the field of lipid nanoparticle (LNP)-based delivery systems for the delivery of nucleic acid, especially for the delivery of mRNA.

### BACKGROUND ART

Progress in mRNA technologies and lipid nanoparticle (LNP)-based delivery systems has allowed the development of mRNA COVID-19 vaccines at unprecedented speed, demonstrating the clinical potential of lipid nanoparticle-mRNA formulations and providing a powerful tool against the SARS-COV-2 pandemic.

LNPs are nucleic acid delivery systems that usually comprises four types of lipids: a sterol (e.g., cholesterol), a helper lipid (typically a phospholipid), a polyethylene glycol (PEG)-lipid, and a cationic/ionizable lipid.

Although the exact structure of LNPs is unknown, it is believed that the cationic/ionizable lipid interacts with the anionic groups of the nucleic acid during the formation of LNPs. When the LNPs enter the cells by endocytosis, cationic/ionizable lipids disturb the negatively charged endosome membrane and allow the release of the nucleic acid into the cytoplasm. Once released in the cytoplasm, the nucleic acid can be active: for example, if the nucleic acid is an mRNA, it can be translated into a protein of interest. If the nucleic acid is an siRNA, it can silence a gene of interest.

In addition to cationic/ionizable lipids, LNPs typically contain other lipid components, such as helper lipids (typically phospholipids), sterols (e.g., cholesterol) and/or polyethylene glycol (PEG)-functionalized lipids (PEG-lipids). These lipids can improve nanoparticle properties, such as particle stability, delivery efficacy, tolerability and biodistribution.

Sterols (e.g., cholesterol) adjust the flexibility and fusogenicity of lipids during mixing, facilitating the LNP formation. It can enhance particle stability by modulating membrane integrity and rigidity. The molecular geometry of the sterols can further affect delivery efficacy and biodistribution of lipid nanoparticles.

The helper lipid is related to LNP structure, interfacial tension, and helps nucleic acid release. For example, 1,2-distearoyl-sn-glycero-3-phosphocholine (DSPC) can form a lamellar phase, which stabilizes the structure of lipid nanoparticles. 1,2-dioleoyl-sn-glycero-3-phosphoethanolamine (DOPE) tends to adopt an inverted hexagonal H (II) phase, which destabilizes endosomal membranes and facilitates endosomal escape of lipid nanoparticles.

PEG-lipids can have multiple effects on the properties of lipid nanoparticles. It can affect particle size and zeta potential. PEG-lipids can further contribute to particle stability by decreasing particle aggregation, and certain PEG modifications prolong the blood circulation time of nanoparticles by reducing clearance mediated by the kidneys and the mononuclear phagocyte system. Finally, PEG-lipids can be used to conjugate specific ligands to the particle for targeted delivery.

LNP-nucleic acid formulations are commonly manufactured by rapid mixing: an organic phase comprising the lipid components and an aqueous phase comprising the nucleic acid are mixed under specific conditions (that is, pH and flow rate). This technique allows reproducible and scalable production of LNP-nucleic acid formulations that show good encapsulation efficiency and homogeneous size distribution.

LNP-nucleic acid formulations are further purified to remove organic solvents and residual components, and the final nucleic acid concentration can be further increased by enrichment. LNP-nucleic acid formulations are then typically filtered and frozen.

A variety of lipid nanoparticles have been explored and optimized for nucleic acid delivery, providing valuable information for the future design of nucleic acid-based therapeutics. However, there are still substantial challenges to overcome.

A first main challenge lies in stability. Despite these merits of efficacy and safety, instability and ultracold storage requirement of LPN-nucleic acid formulations remain major limitations. The stability of this emerging and fast-growing technology is poorly understood, and it likely depends on multiple factors, such as excipients, pH, and temperature.

A second important challenge lies in bioavailability. To function in vivo, LNP-nucleic acid formulations need to overcome multiple extracellular and intracellular barriers. First, the nucleic acid needs to be protected from nuclease degradation in physiological fluids. Second, the formulation should evade the interception by the mononuclear phagocyte system and clearance by renal glomerular filtration post systemic administration. Third, LNP-nucleic acid systems need to reach target tissues, followed by internalization by target cells. Finally, nucleic acid molecules must escape endosomes to reach the cytoplasm. Regarding mRNA-LNPs, it is estimated that less than 2% of the mRNA escapes the endosomes. Furthermore, the mRNA trapped inside endosomes can get degraded over time which can cause cytotoxicity. Therefore, the dose to be administered is today quite limited.

### Technical Problem

It was an object of the invention to provide improved LNP-based delivery systems for the delivery of nucleic acids, preferably mRNAs.

It was another object of the invention to provide LNP-based delivery systems for the delivery of nucleic acids having improved potency and/or improved bioavailability, e.g., allowing to get higher transfection of the nucleic acid, and/or higher release of the nucleic acid into the cytoplasm, and/or higher protein expression level.

It was another object of the invention to provide LNP-based delivery systems for the delivery of nucleic acids having satisfying stability.

It was another object of the invention to solve the abovementioned problems by providing delivery systems having, moreover, the other properties required for these types of products, notably in terms of safety.

### Presentation of the Invention

The inventors developed an improved LNP for the delivery of nucleic acids characterized by the fact that they include a cyclodextrin compound that is nonionic or cationic.

Cyclodextrins are cyclic oligosaccharides which typically come from the enzymatic degradation of starch. The three most common natural cyclodextrins are made up of 6, 7 or 8 α-D-glucopyranose units in chair configuration, linked to one another by α-1,4 bonds. They are more commonly referred to as α, β, or γcyclodextrin, respectively. Their three-dimensional structure appears in the form of a truncated cone on the outside of which are the hydroxyl groups representing the highly hydrophilic part of cyclodextrins. The interior of the cone or the cavity of cyclodextrins is made up of the hydrogen atoms borne by the C3 and C5 carbons and also of the oxygen atoms which participate in the glycosidic bond, thus conferring on them a nonpolar nature.

While one would have expected that including a cyclodextrin in the LNPs would have impaired their structure and function by interacting with the lipids composing them, it was found, on the contrary, that such LNPs had higher potency. The transfection efficiency was improved and better maintained over the time (Examples sections II.A.2., II.B.3, and II.C.3), as compared to LNPs not including any cyclodextrin. Without being bound by any theory, the inventor(s) believe(s) that the LNPs according to the disclosure might transfect into the cells in a greater extent and/or exhibit higher endosomal escape.

Excellent results were obtained with various cyclodextrins (Examples section II.B.), namely methyl-cyclodextrin, hydroxypropyl-cyclodextrin and unsubstituted cyclodextrin). As a comparison, the use of anionic cyclodextrin (sulfobutylether-β-cyclodextrin) impaired the LNPs potency. Without being bound by any theory, the inventors believe that some detrimental interaction might occur with the nucleic acid that bears anionic phosphate groups.

The technical solution according to the disclosure has a broad range of applications: it can be applied to different cell types and to different lipid systems (Examples section II.).

The higher potency and stability of the LNPs according to the disclosure may offer the possibility to decrease the doses of mRNA-LNPs to be administered.

The use of cyclodextrins in LNPs based formulation has been described in the literature (e.g., WO2012170889). However, they were used as cryoprotectants in the lyophilization media, and were not part of the structure of the LNPs. As it is apparent from the Examples section III. herein after, the inventor(s) found that such use was detrimental to the LNPs potency.

### BRIEF DESCRIPTION OF THE INVENTION

The invention first relates to a composition of matter comprising lipid nanoparticles (LNPs), said lipid nanoparticles comprising lipids, a nucleic acid, and a nonionic or cationic cyclodextrin compound, said nucleic acid and cyclodextrin compound being incorporated into the nanoparticle structure comprising said lipids. Preferably, said cyclodextrin compound is nonionic. Preferably, said cyclodextrin compound is selected from a native cyclodextrin, a substituted cyclodextrin, or from a mixture thereof. Preferably, said substituted cyclodextrin is a cyclodextrin substituted with one or more alkyl and/or hydroxyalkyl groups. Preferably, said alkyl and/or hydroxyalkyl groups have 1 to 5 carbon atoms. Preferably, said cyclodextrin compound is selected from a native cyclodextrin, a hydroxypropyl-cyclodextrin, a methyl-cyclodextrin, or from a mixture thereof. Preferably, said lipids comprise a cationic or a cationizable lipid. Preferably, said lipids comprise a sterol. Preferably, said lipids comprise a polyethylene glycol (PEG)-lipid and/or a surfactant other than said (PEG)-lipid. Preferably, said lipids comprise a helper lipid. Preferably, said lipids comprise (a) a cationic or a cationizable lipid, (b) a sterol, (c) a (PEG)-lipid and/or a surfactant other than said (PEG)-lipid, and (d) a helper lipid. Preferably, said sterol is selected from cholesterol, C-24 alkyl steroids, or any mixture thereof. Preferably, said C-24 alkyl steroids are selected from β-sitosterol, stigmasterol, or from any mixture thereof. Preferably, the nitrogen: phosphate (N: P) molar ratio of said LNPs is equal to or higher than 1:1. Preferably, said LNPs have an average diameter equal to or lower than 1000 nm, said average diameter being determined by dynamic light scattering (DLS). 30

The invention also related to a process for making LNPs, comprising putting into contact lipids, a nucleic acid and a nonionic or cationic cyclodextrin compound, so as to obtain LNPs. The invention also relates to LNPs obtainable from this process. The invention also relates to a composition of matter comprising said LNPs.

The invention also relates to a composition of matter according to the disclosure for use as a medicine. The invention also relates to a composition of matter according to the disclosure for use as a vaccine. The invention also relates to a composition of matter according to the disclosure for use in cell therapy. The invention also relates to a composition of matter according to the disclosure for use in gene therapy. The invention also relates to a composition of matter according to the disclosure for use in CRISPR-Cas9 based therapies. The invention also relates to a composition of matter according to the disclosure for use in theranostic applications.

The invention also relates to a method for improving the potency of LNPs, comprising incorporating a nonionic or cationic cyclodextrin compound in said LNPs. The invention also relates to a method for increasing the quantity of protein produced from the nucleic acid of LNPs, comprising incorporating a nonionic or cationic cyclodextrin compound in said LNPs. The invention also relates to a method for increasing the release of the nucleic acid of LNPs into the cell cytoplasm, comprising incorporating a nonionic or cationic cyclodextrin compound in said LNPs. The invention also relates to a method for increasing endosomal escape of the nucleic acid of LNPs, comprising incorporating a nonionic or cationic cyclodextrin compound in said LNPs. The invention also relates to a method for increasing the stability of LNPs, comprising incorporating a nonionic or cationic cyclodextrin compound in said LNPs.

The invention also relates to a method for improving the potency of LNPs, and/or for increasing the quantity of protein produced from the nucleic acid of LNPs, and/or for increasing the release of the nucleic acid of LNPs into the cell cytoplasm, and/or for increasing endosomal escape of the nucleic acid of LNPs, and/or for increasing the stability of LNPs, comprising putting into contact lipids, a nucleic acid and a nonionic or cationic cyclodextrin compound, so as to obtain said LNPs.

### BRIEF DESCRIPTION OF DRAWINGS

Other features, details and advantages will be shown in the following detailed description and on the figures, on which:

FIG. 1 shows the transfection efficiency of mRNA-LNPs obtained from Lipid Composition 1, including or not a cyclodextrin compound, at Day 0 (TO) following their preparation, on HEK cells transfected with 50 ng or 70 ng of mRNA.

FIG. 2 shows the transfection efficiency of mRNA-LNPs obtained from Lipid Composition 1 including or not a cyclodextrin compound, upon 8-day storage at 4° C., on HEK cells transfected with 50 ng or 70 ng of mRNA.

FIG. 3 shows the encapsulation efficiency of mRNA-LNPs obtained from Lipid Composition 2 including or not a cyclodextrin compound, at Day 0 (TO) following their preparation (table), and upon 3-week storage at 4° C. or 25° C. (graphs).

FIG. 4 shows the average diameter of mRNA-LNPs obtained from Lipid Composition 2 including or not a cyclodextrin compound, at Day 0 (TO) following their preparation (table), and upon 2-week storage at 4° C. or 25° C. (graphs).

FIG. 5 shows the transfection efficiency of mRNA-LNPs obtained from Lipid Composition 2 including or not a cyclodextrin compound, upon 20-day storage at 4° C. or 25° C., on HEK cells.

FIG. 6 shows the transfection efficiency of mRNA-LNPs obtained from Lipid Composition 2 including or not a cyclodextrin compound, upon 20-day storage at 4° C. or 25° C., on Hela cells.

FIG. 7 shows the encapsulation efficiency and average diameter of mRNA-LNPs obtained from Lipid Composition 3 including or not a cyclodextrin compound, at Day 0 (T0) following their preparation.

FIG. 8 shows the transfection efficiency of mRNA-LNPs obtained from Lipid Composition 3 including or not a cyclodextrin compound, upon 7-day storage at 4° C. or 25° C., on Hela cells.

FIG. 9 shows the transfection efficiency of cyclodextrin-free mRNA-LNPs suspended in a buffer comprising or not a cyclodextrin compound, at Day 0 (TO) following their preparation.

FIG. 10 is one hypothetical representation of a lipid nanoparticle (not incorporating a cyclodextrin compound).

### DESCRIPTION OF EMBODIMENTS

Figures and the following detailed description contain, essentially, some exact elements. They can be used to enhance understanding the invention and, also, to define the invention if necessary.

### Composition of Matter Comprising Lipid Nanoparticles (LNPs)

The invention first relates to a composition of matter comprising lipid nanoparticles (LNPs), said lipid nanoparticles comprising lipids, a nucleic acid, and a nonionic or cationic cyclodextrin compound, said nucleic acid and cyclodextrin compound being incorporated into the nanoparticle structure comprising said lipids.

Lipid nanoparticles (LNPs) are nucleic acid delivery systems that generally comprise four types of lipids: a sterol (e.g., cholesterol), a helper lipid (e.g., a phospholipid), a polyethylene glycol (PEG)-lipid, and a cationic/cationizable lipid. Examples of such lipids are given in the article Hou, X et al., Lipid nanoparticles for mRNA delivery. Nature Reviews Materials, 6(12), 1078-1094 (2021). Instead of polyethylene glycol (PEG)-lipids, or further to polyethylene glycol (PEG)-lipids, other types of surfactants may be used. These are typically non-ionic surfactants e.g., polysorbates (e.g., TWEEN® Series), Polyoxyethylene (POE) fatty ethers (e.g., Brij® Series), polyoxyethylene esters of 12-hydroxystearic acid (e.g., Solutol), Polyoxyethylene (40) stearate (Myrj52).

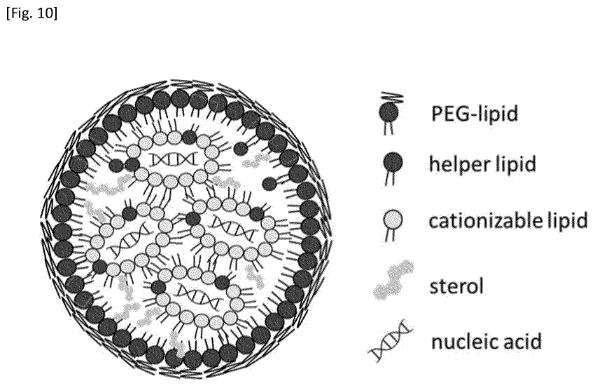

LNPs are typically sized on the order of micrometers or smaller. The nucleic acid to be delivered is incorporated in the LNPs. Insofar as the nature of the association between the lipids and the nucleic acid is only hypothetical, the term “incorporated” is intended to mean “associated with”, whether inside the nanoparticles formed by the lipids, or on its surface. That being said, it is likely that the nucleic acid is essentially located inside (i.e., encapsulated in) the LNPs (see FIG. 10, which is one example of the hypothetical structure of a LNP). When referring to LNPs, the term “nucleic acid-LNPs” may also be employed. When referring to LNPs in which the nucleic acid is a mRNA, the term “mRNA-LNPs” may be employed. When referring to LNPs in which the nucleic acid is a siRNA, the term “siRNA-LNP” may be employed etc. The LNPs according to the disclosure also incorporate a cyclodextrin compound. Similarly, the nature of the association between the cyclodextrin compound and the other constituents of the LNPs is hypothetical, such that the expression “incorporated” is intended to mean “associated with”, whether inside the nanoparticles formed by the lipids, or on its surface. It should be noted that in the case of the cyclodextrin compound, it cannot be verified to what extent it is incorporated in the LNPs, as there are no good methods available for quantifying cyclodextrins. However, in view of the negative results obtained when the cyclodextrin compound is added outside the LNPs (i.e., not incorporated in the LNPs), it is very likely that the cyclodextrin compound is at least partially incorporated in the LNPs. Without being bound by any theory, the inventors believe that the cyclodextrin compound interacts with the nucleic acid of the LNPs.

### Lipids

The lipids of the LNPs according to the disclosure typically comprise a cationic lipid and/or a cationizable lipid.

The expression “cationic lipid” classically refers to a lipid that is positively charged at any pH, more specifically, at physiological and endosomal pH. The expression “cationizable lipid” classically refers to a lipid which is neutral at physiological pH and positively charged at lower pH, in particular at endosomal pH. Typically, these cationizable lipids are protonated at acidic pH.

Typically, the cationic/cationizable lipids according to the disclosure bear more cationic or cationizable groups than anionic groups. Still preferably, they don't bear any anionic group.

Preferably, the cationic/cationizable lipid according to the disclosure is a cationizable lipid. The pH-sensitivity of cationizable lipids is beneficial for nucleic acid delivery in vivo, because neutral lipids have less interactions with the anionic membranes of blood cells and, thus, improve the biocompatibility of lipid nanoparticles. Trapped in endosomes, in which the pH is lower than in the extracellular environment, cationizable lipids are protonated and, therefore, become positively charged, which may promote membrane destabilization and facilitate endosomal escape of the LNPs.

Non-limiting examples of cationic/cationizable lipids which can be used are:
1,2-di-O-octadecenyl-3-trimethylammonium-propane (DOTMA);
1,2-dioleoyl-3-trimethylammonium-propane (DOTAP);
Dimethyldioctadecylammonium bromide (DDAB);
2,3-dioleyloxy-N-[2-(sperminecarboxamido)ethyl]-N,N-dimethyl-1-propanaminium trifluoroacetate (DOSPA);
2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide (BHEM-cholesterol);
Ethylphosphatidylcholine (ePC);
(2S)-2,5-bis(3-aminopropylamino)-N-[2-(dioctadecylamino) acetyl] pentanamide (DOGS; Transfectam);
N1-[2-((1S)-1-[(3-aminopropyl)amino]-4-[di(3-aminopropyl)amino]butylcarboxamido)ethyl]-3,4-di[oleyloxy]-benzamide (MVL5);
DC-Cholesterol;
N4-cholesteryl spermine (GL67);
1,2-dilinoleyloxy-N,N-dimethyl-3-aminopropane (DLin-DMA);
2,2-dilinoleyl-4-(2-dimethylaminoethyl)-[1,3]-dioxolane (DLin-KC2-DMA);
(6Z,9Z,28Z,31Z)-heptatriaconta-6,9,28,31-tetraen-19-yl 4-(dimethylamino)butanoate (DLin-MC3-DMA);
di((Z)-non-2-en-1-yl) 9-((4-(dimethylamino)butanoyl)oxy) heptadecanedioate (L319);
heptadecan-9-yl 8-((2-hydroxyethyl) (8-(nonyloxy)-8-oxooctyl)amino) octanoate (Lipid 5);
heptadecan-9-yl 8-((2-hydroxyethyl) (6-oxo-6-(undecyloxy) hexyl)amino) octanoate (Lipid H (SM-102));
((4-hydroxybutyl) azanediyl)bis(hexane-6,1-diyl)bis(2-hexyldecanoate) (ALC-0315);
1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200);
tetrakis (8-methylnonyl) 3,3′,3″,3″-(((methylazanediyl)bis(propane-3,1 diyl))bis(azanetriyl)) tetrapropionate (306Oᵢ₁₀);
3,6-bis(4-(bis(2-hydroxydodecyl)amino)butyl) piperazine-2,5-dione (cKK-E12);
3,6-bis(4-(bis ((9Z,12Z)-2-hydroxyoctadeca-9,12-dien-1-yl)amino)butyl) piperazine-2,5-dione (OF-02);
(((3,6-dioxopiperazine-2,5-diyl)bis(butane-4,1-diyl))bis(azanetriyl)) tetrakis (ethane-2,1-diyl) (9Z,9′Z,9″Z,9″Z,12Z,12′Z,12″Z,12′″Z)-tetrakis (octadeca-9,12-dienoate) (OF-Deg-Lin);
(((3,6-dioxopiperazine-2,5-diyl)bis(butane-4,1-diyl))bis(azanetriyl)) tetrakis (butane-4,1-diyl) (9Z,9′Z,9″Z,9″Z,12Z,12′Z,12″Z,12″Z)-tetrakis (octadeca-9,12-dienoate) (OF-C4-Deg-Lin);
N¹,N³,N⁵-tris(3-(didodecylamino) propyl)benzene-1,3,5-tricarboxamide (TT3);
Hexa (octan-3-yl) 9,9′,9″,9″,9″″,9′″″-((((benzene-1,3,5-tricarbonyl)ris(azanediyl)) tris (propane-3,1-diyl)) tris (azanetriyl)) hexanonanoate (FTT5);
   the one use in product GenVoy-ILM™.

Preferably the cationic/cationizable lipid according to the disclosure comprises, or consists in: the one use in product GenVoy-ILM™, ALC-0315, SM-102, DLin-MC3-DMA, or any mixture thereof.

Preferably, the total amount of cationic/cationizable lipid of the LNPs according to the disclosure is equal to or higher than 30% molar, said percentage being expressed with respect to the total amount of lipids composing the LNPs. It is preferably equal to or higher than 35%, preferably equal to or higher than 40%, preferably equal to or higher than 45%. It is preferably equal to or lower than 80%, preferably equal to or lower than 70%, preferably equal to or lower than 65%, preferably equal to or lower than 60%, preferably equal to or lower than 55%. It is for example equal to about 50%.

The lipids of the LNPs according to the disclosure typically comprise a sterol. Non-limiting examples of sterols are cholesterol, fecosterol, sitosterol, ergosterol, campesterol, stigmasterol, brassicasterol, tomatidine, tomatine ursolic acid, alpha-tocopherol, cholesteryl oleate, or any mixture thereof.

The sterol can be cholesterol. It can also be selected from cholesterol derivatives or cholesterol analogues, as long as it does not interfere with the desired properties of the LNPs, notably in terms of efficacy and safety. Preferably, the sterol according to the disclosure comprises, or consists in: cholesterol, C-24 alkyl steroids (e.g., β-sitosterol, stigmasterol), or any mixture thereof. Preferably, the sterol according to the disclosure comprises, or consists in: cholesterol, β-sitosterol, stigmasterol, or any mixture thereof, still preferably cholesterol.

Preferably, the amount of sterol of the LNPs according to the disclosure is equal to or higher than 0.1% molar, said percentage being expressed with respect to the total amount of lipids composing the LNPs. It is preferably equal to or higher than 0.5%, preferably equal to or higher than 1.0%, preferably equal to or higher than 1%, preferably equal to or higher than 5%, preferably equal to or higher than 10%, preferably equal to or higher than 20%, preferably equal to or higher than 30%, preferably equal to or higher than 35%. It is preferably equal to or lower than 60%, preferably equal to or lower than 50%, preferably equal to or lower than 40%. It is for example equal to about 37.5%, or equal to about 38%, or equal to about 38.5%, or equal to about 39%.

The lipids of the LNPs according to the disclosure typically comprise a PEG-lipid, which is a lipid functionalized with polyethylene glycol (PEG), and/or a surfactant other than said PEG-lipid.

Non-limiting examples of PEG-lipids include PEG-modified phosphatidylethanolamines, PEG-modified phosphatidic acids, PEG-modified ceramides, PEG-modified dialkylamines, PEG-modified dialkylglycerols, PEG-modified diacylglycerols, PEG-modified 1,2-diacyloxypropan-3-amines, PEG-modified fatty acids, or any mixture thereof. Such lipids are also commonly referred to as PEGylated lipids.

Preferably, the PEG-lipid is selected from 1,2-dimyristoyl-sn-glycerol-3-methoxypolyethylene glycol (PEG-DMG), 1,2-distearoyl-sn-glycero-3-phosphoethanolamine-N-[amino (polyethylene glycol)] (PEG-DSPE), 1,2-Distearoyl-rac-glycero-3-methoxypolyethyleneglycol (PEG-DSG), 1,2-dipalmitoyl-sn-glycero-3-phosphoethanolamine-N-methoxypolyethyleneglycol (PEG-dipalmitoyl), PEG-dioleoyl, Distearoyl-rac-glycerol-polyethyleneglycol (PEG-distearyl), Methoxy-polyethyleneglycol-diacylglycerol (PEG-DAG), N-(Methylpolyoxyethylene oxycarbonyl)-1,2-dipalmitoyl-sn-glycero-3-phosphoethanolamine (PEG-DPPE), or PEG-1,2-dimyristyloxlpropyl-3-amine (PEG-c-DMA), R-3-[(ω-methoxypolyethyleneglycol) carbamoyl]-1,2-dimyristyloxy-propyl-3-amine (PEG-c-DOMG), Methoxy-polyethyleneglycol-1,2-Dilauroyl-sn-Glycero-3-Phosphoethanolamine (PEG-DLPE), 1,2-dimyristoyl-sn-glycero-3-phosphoethanolamine-N-methoxypolyethylene glycol (PEG-DMPE), 1,2-dihexadecanoyl-sn-glycero-3-phosphocholine-N-methoxypolyethyleneglycol (PEG-DPPC), 2-[(polyethyleneglycol)-2000]-N,N-ditetradecylacetamide (ALC-0159), or any mixture thereof.

Preferably, the PEG-lipid according to the disclosure comprises or consists in PEG-DMG.

In some embodiments, the lipid moiety of the PEG-lipids includes those having lengths of from about C₁₄ to about C₂₂, preferably from about C₁₄ to about C₁₆. In some embodiments, a PEG moiety, for example a mPEG-NH2, has a size of about 1000, 2000, 5000, 10 000, 15 000 or 20 000 daltons. It is preferably of about 2000 daltons. Preferably, the PEG-lipid is PEG2k-DMG.

Preferably, the PEG-lipid according to the disclosure comprises, or consists in: polyethylene glycol 2000 dimyristoyl glycerol (PEG2k-DMG), 1,2-distearoylrac-glycero-3-methoxypolyethylene glycol-2000 (PEG2k-DSG), or any mixture thereof. Preferably, the PEG-lipid according to the disclosure comprises or is PEG2k-DMG.

Preferably, the amount of PEG-lipid according to the disclosure is equal to or higher than 0.1% molar, said percentage being expressed with respect to the total amount of lipids composing the LNPs. It is preferably equal to or higher than 0.5%, preferably equal to or higher than 0.8%, preferably equal to or higher than 1.0%, preferably equal to or higher than 1.1%, preferably equal to or higher than 1.2%, preferably equal to or higher than 1.3%, preferably equal to or higher than 1.4%, preferably equal to or higher than 1.5%. It is preferably equal to or lower than 10.0%, preferably equal to or lower than 5.0%, preferably equal to or lower than 4.0%, preferably equal to or lower than 3.5%, preferably equal to or lower than 3.0%, preferably equal to or lower than 2.9%, preferably equal to or lower than 2.8%, preferably equal to or lower than 2.7%, preferably equal to or lower than 2.6%, preferably equal to or lower than 2.5%. It is for example equal to about 1.5%, or equal to about 2.5%.

Non-limiting examples of surfactants other than the PEG-lipid according to the disclosure are polysorbates (e.g., TWEEN® Series) such as polysorbate 20 (e.g., TWEEN® 20), polysorbate 40 (e.g., TWEEN® 40), polysorbate 60 (e.g., TWEEN® 60), polysorbate 80 (e.g., TWEEN® 80), D-a-tocopherol polyethylene glycol succinate (TPGS), Polyoxyethylene (POE) fatty ethers (e.g., Brij® Series), polyoxyethylene esters of 12-hydroxystearic acid (e.g., Solutol), Polyoxyethylene (40) stearate (Myrj52), or a mixture thereof.

Preferably, the amount surfactant other than the PEG-lipid according to the disclosure is equal to or higher than 0.01% molar, said percentage being expressed with respect to the total amount of lipids composing the LNPs. It is preferably equal to or higher than 0.05%, preferably equal to or higher than 0.1%, preferably equal to or higher than 0.5%, preferably equal to or higher than 0.8%, preferably equal to or higher than 1.0%, preferably equal to or higher than 1.1%, preferably equal to or higher than 1.2%, preferably equal to or higher than 1.3%, preferably equal to or higher than 1.4%, preferably equal to or higher than 1.5%. It is preferably equal to or lower than 10.0%, preferably equal to or lower than 5.0%, preferably equal to or lower than 4.0%, preferably equal to or lower than 3.5%, preferably equal to or lower than 3.0%, preferably equal to or lower than 2.9%, preferably equal to or lower than 2.8%, preferably equal to or lower than 2.7%, preferably equal to or lower than 2.6%, preferably equal to or lower than 2.5%. It is for example equal to about 1.5%, or equal to about 2.5%.

The lipids of the LNPs according to the disclosure typically comprise a helper lipid i.e., a lipid contributing to the stability and delivery efficiency of the LNP, and/or a lipid able to bind the receptor apolipoprotein E (ApoE), said ApoE receptor being preferably a mammal ApoE receptor, still preferably a human ApoE receptor. Preferably, the helper lipid according to the disclosure is selected from phospholipids, preferably from phosphatidylcholines or phosphatidylethanolamines, or from any mixture thereof.

In general, phospholipids include a phospholipid moiety and one or more fatty acid moieties. A phospholipid moiety may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid.

Non-limiting examples of phospholipids include:
1,2-distearoyl-sn-glycero-3-phosphocholine (DSPC);
1,2-dioleoyl-sn-glycero-3-phosphoethanolamine (DOPE);
1,2-dilinoleoyl-sn-glycero-3-phosphocholine (DLPC);
1,2-dimyristoyl-sn-glycero-phosphocholine (DMPC);
1,2-dioleoyl-sn-glycero-3-phosphocholine (DOPC);
1,2-dipalmitoyl-sn-glycero-3-phosphocholine (DPPC);
1,2-diundecanoyl-sn-glycero-phosphocholine (DUPC);
1-palmitoyl-2-oleoyl-sn-glycero-3-phosphocholine (POPC);
1,2-di-O-octadecenyl-sn-glycero-3-phosphocholine (18:0 Diether PC);
1-oleoyl-2-cholesterylhemisuccinoyl-sn-glycero-3-phosphocholine (OChemsPC);
1-hexadecyl-sn-glycero-3-phosphocholine (C16 Lyso PC);
1,2-dilinolenoyl-sn-glycero-3-phosphocholine;
1,2-diarachidonoyl-sn-glycero-3-phosphocholine;
1,2-didocosahexaenoyl-sn-glycero-3-phosphocholine;
1,2-diphytanoyl-sn-glycero-3-phosphoethanolamine (ME 16.0 PE);
1,2-distearoyl-sn-glycero-3-phosphoethanolamine;
1,2-dilinoleoyl-sn-glycero-3-phosphoethanolamine;
1,2-dilinolenoyl-sn-glycero-3-phosphoethanolamine;
1,2-diarachidonoyl-sn-glycero-3-phosphoethanolamine;
1,2-didocosahexaenoyl-sn-glycero-3-phosphoethanolamine;
1,2-dioleoyl-sn-glycero-3-phospho-rac-(1-glycerol) sodium salt (DOPG); sphingomyelin;
   or any mixture thereof.

Preferably, the helper lipid according to the disclosure comprises, or consists in: DSPC, DOPE or a mixture thereof.

Preferably, the amount of helper lipid of the LNPs according to the disclosure is equal to or higher than 2% molar, said percentage being expressed with respect to the total amount of lipids composing the LNPs. It is preferably equal to or higher than 5%, preferably equal to or higher than 6%, preferably equal to or higher than 7%, preferably equal to or higher than 8%, preferably equal to or higher than 9%. It is preferably equal to or lower than 30%, preferably equal to or lower than 20%, preferably equal to or lower than 15%, preferably equal to or lower than 14%, preferably equal to or lower than 13%, preferably equal to or lower than 12%, preferably equal to or lower than 11%. It is for example equal to about 10%.

Preferably, the lipids of the LNPs according to the disclosure comprise or consist in:
a cationic/cationizable lipid, preferably as described before;
a sterol, preferably as described before;
a PEG-lipid and/or a surfactant other that said PEG-lipid, preferably as described before;
a helper lipid (preferably a phospholipid), preferably as described before.

### Cyclodextrin Compound

The LNPs according to the disclosure further comprise a nonionic or cationic cyclodextrin compound, i.e., a cyclodextrin compound whose net charge is neutral or positive respectively i.e., a cyclodextrin compound that is not anionic. Preferably, the cyclodextrin compound according to the disclosure doesn't bear any anionic group. Preferably, the cyclodextrin compound according to the disclosure is nonionic i.e., neutral. Preferably, the nonionic cyclodextrin compound according to the disclosure doesn't bear any ionic group.

The expression «cyclodextrin compound» classically refers to a mixture of cyclodextrin molecules, further comprising the residual substances resulting from its manufacturing process. Such cyclodextrin compound might be a native or a substituted cyclodextrin. Contrary to chemical substances with well-defined structure, substituted cyclodextrins generally are a mixture of substituted cyclodextrin molecules having different substitution patterns, and which are thus structurally different.

It is understood that a cyclodextrin compound is different from a cyclodextrin polymer, the latter being composed of cyclodextrin molecules covalently bound to one another.

In a first embodiment, the nonionic cyclodextrin compound according to the disclosure is a native (i.e., unsubstituted) cyclodextrin, preferably a beta-cyclodextrin.

In a second preferred embodiment, the nonionic cyclodextrin compound according to the disclosure is substituted. It is preferably substituted with alkyl group and/or hydroxyalkyl group, preferably having 1 to 5 carbon atoms, preferably 1 to 4, preferably 1 to 3, preferably 1 or 3. It is preferably selected from hydroxypropyl-cyclodextrin, methyl-cyclodextrin, or from a mixture thereof. It is preferably selected from hydroxypropyl-β-cyclodextrin (HPBCD), methyl-β-cyclodextrin (MBCD), or from a mixture thereof.

Preferably, the substituted nonionic cyclodextrin compound according to the disclosure has an average molar substitution degree (MS) equal to or higher than 0.10, preferably equal to or higher than 0.20, preferably equal to or higher than 0.30, preferably equal to or higher than 0.40, preferably equal to or higher than 0.50, preferably equal to or higher than 0.58. Preferably, this MS is equal to or lower than 1.50, preferably equal to or lower than 1.40, preferably equal to or lower than 1.30, preferably equal to or lower than 1.20, preferably equal to or lower than 1.10, preferably equal to or lower than 1.00, preferably equal to or lower than 0.99.

It is reminded that the “average molar substitution degree (MS)” refers to the average number of added substituents per anhydroglucose unit. It should be noted that this MS is different from the average molecular substitution degree (DS), which refers to the average number of added substituents per cyclodextrin molecule, and which is, as a consequence, function of the number of anhydroglucose units forming the original cyclodextrin. For instance, for alkylated beta-cyclodextrins, this DS is equal to 7 times the MS, as the β-cyclodextrins are made of 7 anhydroglucose units.

In a preferred embodiment, the alkylated nonionic cyclodextrin compound according to the disclosure is hydroxypropyl-cyclodextrin, preferably HPBCD.

Preferably, the hydroxypropyl-cyclodextrin according to the disclosure (e.g., HPBCD), has an average molar substitution degree (MS) as described before. The MS of the hydroxypropyl-cyclodextrin according to the disclosure (e.g., HPBCD), can classically be determined by the person skilled in the art by proton nuclear magnetic resonance (NMR), preferably according to the USP 41 NF 36 method «Hydroxypropyl Betadex; Molar substitution».

In a first preferred embodiment, the MS of the hydroxypropyl-cyclodextrin according to the disclosure (e.g., HPBCD) is equal to or higher than 0.60, preferably equal to or higher than 0.70, preferably equal to or higher than 0.80, preferably equal to or higher than 0.81. It is preferably equal to or lower than 0.99. Such hydroxypropyl-cyclodextrin is commercially available. For example, mention can be made of KLEPTOSE® HP (ROQUETTE FRERES).

In a second preferred embodiment, the MS of the hydroxypropyl-cyclodextrin according to the disclosure (e.g., HPBCD) is equal to or higher than 0.55, preferably equal to or higher than 0.58. It is preferably equal to or lower than 0.90, preferably equal to or lower than 0.80, preferably equal to or lower than 0.70, preferably equal to or lower than 0.68. Such hydroxypropyl-cyclodextrin is commercially available. For example, mention can be made of KLEPTOSE® HPB (ROQUETTE FRERES).

In a third preferred embodiment, the MS of the hydroxypropyl-cyclodextrin according to the disclosure (e.g., HPBCD) is equal to or higher than 0.50, preferably equal to or higher than 0.55, preferably equal to or higher than 0.58. It is preferably equal to or lower than 0.90, preferably equal to or lower than 0.80, preferably equal to or lower than 0.71. Such hydroxypropyl-cyclodextrin is commercially available. For example, mention can be made of KLEPTOSE® HPB-LB (ROQUETTE FRERES).

Preferably, the hydroxypropyl-cyclodextrin according to the disclosure (e.g., HPBCD), has the following substitution profile, as determined by electrospray ionization—Mass spectrometry (ESI-MS):
signal corresponding to unsubstituted cyclodextrin (HP0): equal to or lower than 5%, preferably equal to or lower than 4%, preferably equal to or lower than 3%, preferably equal to or lower than 2%, preferably equal to or lower than 1%, preferably equal to 0%; and/or,
signal corresponding to the hydroxypropyl-cyclodextrin molecules having 1 substitution (HP1): equal to or lower than 5%, preferably equal to or lower than 4%, preferably equal to or lower than 3%, preferably equal to or lower than 2%, preferably equal to or lower than 1%; and/or,
signal corresponding to the hydroxypropyl-cyclodextrin molecules having 2 substitutions (HP2): equal to or lower than 5%, preferably equal to or lower than 4%, preferably equal to or lower than 3%, preferably equal to or lower than 2%; and/or,
signal corresponding to the hydroxypropyl-cyclodextrin molecules having 3 substitutions (HP3): from 0 to 15%, preferably from 0 to 10%, preferably from 0 to 9%, preferably from 0 to 8%, preferably from 0 to 6%; and/or,
signal corresponding to the hydroxypropyl-cyclodextrin molecules having 4 substitutions (HP4): from 0 to 25%, preferably from 0 to 20%, preferably from 0 to 15%, preferably from 1 to 15%; and/or,
signal corresponding to the hydroxypropyl-cyclodextrin molecules having 5 substitutions (HP5): from 0 to 40%, preferably from 0 to 35%, preferably from 0 to 30%, preferably from 1 to 25%; and/or,
signal corresponding to the hydroxypropyl-cyclodextrin molecules having 6 substitutions (HP6): from 0 to 50%, preferably from 1 to 45%, preferably from 3 to 40%, preferably from 5 to 35%, preferably from 10 to 30%; and/or,
signal corresponding to the hydroxypropyl-cyclodextrin molecules having 7 substitutions (HP7): from 5 to 50%, preferably from 10 to 40%, preferably from 15 to 30%; and/or,
signal corresponding to the hydroxypropyl-cyclodextrin molecules having 8 substitutions (HP8): from 1 to 50%, preferably from 2 to 45%, preferably from 3 to 40%, preferably from 4 to 35%, preferably from 5 to 30%; and/or,
signal corresponding to the hydroxypropyl-cyclodextrin molecules having 9 substitutions (HP9): from 0 to 30%, preferably from 1 to 25%; and/or,
signal corresponding to the hydroxypropyl-cyclodextrin molecules having 10 substitutions (HP10): from 0 to 20%, preferably from 0 to 15%, preferably from 0 to 10%; and/or,
signal corresponding to the hydroxypropyl-cyclodextrin molecules having 11 substitutions (HP11): equal to or lower than 10%, preferably equal to or lower than 8%, preferably equal to or lower than 5%, preferably equal to or lower than 3%; and/or,
signal corresponding to the hydroxypropyl-cyclodextrin molecules having 12 substitutions (HP12): equal to or lower than 5%, preferably equal to or lower than 4%, preferably equal to or lower than 3%, preferably equal to or lower than 2%, preferably equal to or lower than 1%; and/or,
signal corresponding to the hydroxypropyl-cyclodextrin molecules having 13 or more substitutions (HP≥13): equal to or lower than 5%, preferably equal to or lower than 4%, preferably equal to or lower than 3%, preferably equal to or lower than 2%, preferably equal to or lower than 1%, preferably equal to 0%;
these percentages being expressed with respect of the sum of the signals obtained for each substitution for which the signal was higher than the background.

It is understood that “signal” refers to the area under the curve of the ion(s) corresponding to the substitutions degree(s) of interest.

In the present disclosure, the substitution profile is determined by ESI-MS, preferably by calculating the average of the measurements performed in triplicate. For determining this substitution profile, it is possible to proceed according to the following method:

-A solution hydroxypropyl-cyclodextrin is prepared at 1 g·L⁻¹ (w/v) in methanol: water (50/50, v/v) with 1 mM sodium acetate. Infusion is performed for 1 min at 10 μL/min and data are recorded. In between two following injections, 500 μL of methanol:water (50/50, v/v) are injected to wash the ion source.
ESI parameters: Spray voltage: 5 KV; Sheath gas: 9; Auxiliary gas: 2; Sweep gas: 0; Spray voltage: 5 KV; Capillary voltage: 23 V; Capillary temperature: 275° C.; Tube lens: 80 V
MS parameters: Full scan; Scan ranges: 50-2000 m/z; Mass range: normal; Scan rate: enhanced; Data acquisition time: 1 min.
For each substituted hydroxypropyl-cyclodextrin species (named as HPX, X being the number of hydroxypropyl substitutions), the corresponding ion current (XIC) is integrated and compared to the sum of all HPX ion currents. As the sodium adduct is the most intense hydroxypropyl-cyclodextrin ion (almost 100 times superior to [M+H]⁺ or [M+NH4]⁺), its peak area is integrated to estimate the proportion of the corresponding HPX molecule.

Preferably, the substitution pattern of the hydroxypropyl-cyclodextrin according to the disclosure (e.g., HPBCD) is such that:
the molar proportion of unsubstituted (no-OHP) moities is from 20 to 70%, preferably from 25 to 60%, preferably from 30 to 50%; and/or,
the molar proportion of C2 substituted (2 OHP) moieties is from 10 to 50%, preferably from 15 to 45%, preferably from 20 to 40%, preferably from 20 to 35%; and/or,
the molar proportion of C3 substituted (3 OHP) moieties is from 2 to 15%, preferably from 4 to 10%, preferably from 6 to 8%; and/or,
the molar proportion of C6 substituted (6 OHP) moieties is from 0.5 to 10%, preferably from 1 to 8%, preferably from 2 to 5%; and/or,
the molar proportion of C2 and C3 substituted (2,3-di-OHP) moieties is from 1 to 20%, preferably from 3 to 15%, preferably from 5 to 15%; and/or,
the molar proportion of C2 and C6 substituted (2,6-di-OHP) moieties is from 0.5 to 15%, preferably from 1 to 10%, preferably from 2 to 10%; and/or,
the molar proportion of twice C3 substituted (3,3′-di-OHP) moieties is equal to or lower than 3%, preferably equal to or lower than 2%, preferably from 0.1 to 2%, preferably from 0.3 to 1.5%; and/or,
the molar proportion of C2, C3 and C6 substituted (2,3,6-tri-OHP) moieties is equal to or lower than 10%, preferably equal to or lower than 5%, for example from from 0.3 to 10%, preferably from 0.5 to 5%; and/or,
the molar proportion of substitutions corresponding to mono-substitutions is from to 90%, preferably from 50 to 85%, preferably from 60 to 80%; and/or,
the molar proportion of substitutions corresponding to di-substitutions is from 5 to 50%, preferably from 10 to 45%, preferably from 15 to 40%, preferably from 20 to 35%; and/or,
the molar proportion of substitutions corresponding to tri-substitutions is from 0.5 to 15%, preferably from 0.5 to 10%, preferably from 1 to 7%, preferably from 1 to 5%; and/or,
the C2/C6 substitutions molar ratio is from 1.0 to 15.0, preferably from 1.5 to 12.0, preferably from 2.0 to 10.0, preferably from 2.5 to 9.0, preferably from 3.0 to 8.0;
and/or,
the C2/C3 substitutions molar ratio is from 0.5 to 5.0, preferably from 1.0 to 4.0, preferably from 1.5 to 3.5, preferably from 2.0 to 3.0.

The percentages correspond to the percentages of anhydroglucose units having the type of substitution considered. For example, for a 2 OHP value equal to 30.0%, it is estimated that 30.0 mol % of the anhydroglucose units of the hydroxypropyl-cyclodextrin are substituted by a hydroxypropyl group at the C2 carbon. As a further example, for a 3,3′-di-OHP value equal to 0.4%, it is estimated that 0.4 mol % of the anhydroglucose units of the hydroxypropyl-cyclodextrin are substituted twice at the C3 carbon (that is to say that the C3 carbon bears two hydroxypropyl groups). As a last example, for a 2,6-di-OHP value equal to 5.0%, it is estimated that 5.0 mol % of the anhydroglucose units of the hydroxypropyl-cyclodextrin are substituted by a hydroxypropyl group both at the C2 carbon, and at the C6 carbon. For the first example, mention will be made of mono-substitution, whereas mention will be made of di-substitution for the last two examples.

Preferably, the hydroxypropyl-cyclodextrin according to the disclosure comprises less than 5.0% of substitutions other than those listed above, for example 3,6—OHP substitutions, preferably less than 4.0%, preferably less 3.0%, preferably less than 2.0%, preferably less than 1.0%, preferably less than 0.5%. Still preferably, the hydroxypropyl-cyclodextrin according to the disclosure comprises no other types of substitutions than those listed above. The expression “no other types of substitutions” is understood to mean that the anhydroglucose units comprising such substitutions are not detectable, in particular by the “Hakomori” method, said method comprising subjecting the hydroxypropyl-cyclodextrin to the following successive steps: permethylation, hydrolysis, reduction, peracetylation.

These substitution patterns can be determined by those skilled in the art, for example according to a method analogous to the “Hakomori” method, typically by subjecting the hydroxypropyl-cyclodextrin to the following successive steps: permethylation, hydrolysis, reduction, peracetylation.

It is possible for example to follow the method as described in U.S. Pat. No. 5,096,893, column 6, example 9 to column 7, example 10 included, said method being incorporated by reference. Typically, the method according to U.S. Pat. No. 5,096,893 is as follows:

Sodium hydride (0.07 mol) is added to anhydrous dimethyl sulfoxide (20 ml) under argon and the mixture is heated for 1 hour at about 60° C. Then, the hydroxypropyl-cyclodextrin (4 g) which has been dried (3 hours at 110° C.) and dissolved in dimethyl sulfoxide (15 ml) is added and placed under argon with stirring at room temperature for 3 hours. The reaction medium is cooled in an ice bath and methyl iodide (10 ml, 0.161 mol) is added dropwise. After another hour in an ice bath, the mixture is left under stirring overnight. Then water (24 ml) is added while cooling and the product is extracted twice with chloroform (90 ml in total). The extract is washed with water (20 ml) and evaporated. The residue is treated with water (25 ml) and extracted three times with ether (75 ml in total). The extract is washed with water and then evaporated. The residue is dissolved in ether (100 ml), then stirred for 30 minutes in the presence of neutral alumina, filtered, then evaporated, until typically 3.7 g of the permethylated product is obtained. 3 mg of the permethylated product is dissolved in aqueous trifluoroacetic acid (0.5 ml), then stored in a screw-cap tube at 100° C. overnight, and concentrated by rinsing with air. The residue and sodium borohydride (100 mg) are dissolved in aqueous ammonia (0.5 ml) and the solution is placed at room temperature for one hour. The solution is acidified with 50% acetic acid (2 drops), and then concentrated. Boric acid is evaporated by co-distillation, first with an acetic acid/methanol mixture (1:9, 5 ml) and then with methanol (25 ml). The residue is treated with acetic anhydride and pyridine (2:1, 0.5 ml) at 100° C. for 30 minutes, concentrated, and separated between chloroform and water (2:1, 6 ml). The chloroform phase is concentrated and the residue is analyzed by gas chromatography and gas-liquid chromatography coupled with mass spectrometry (GLC-MS). The gas-liquid chromatography is performed for example on Hewlett Packard 5830 A equipment fitted with a flame ionization detector, using hydrogen as carrier gas. Gas-liquid chromatography coupled with mass spectrometry is performed for example on a Hewlett Packard 5790-5970 system, using helium as carrier gas. A capillary column (length 25 m, internal diameter 0.20 mm) made of (crosslinked 5% methylphenylsilicone) fused glass, for example of Hewlett Packard Ultra 2 type, is used. The temperature is programmed as follows: 8 minutes at 185° C., ->250° C. at 5° C. per minute, 250° C. for 10 minutes.

In a first preferred embodiment, the hydroxypropyl-cyclodextrin according to the disclosure (e.g., HPBCD) has:
(i) a MS ranging from 0.70 to 1.20, preferably from 0.75 to 1.10, preferably from 0.80 to 1.00, in particular from 0.81 to 0.99; and/or,
(ii) the following substitution profile, as determined by electrospray ionization-Mass spectrometry (ESI-MS):
   HP≤3: equal to or lower than 5%, preferably equal to or lower than 4%, preferably equal to or lower than 3%, preferably equal to or lower than 2%, preferably equal to or lower than 1%; and/or,
   HP4: from 0 to 5%, preferably from 0 to 4%, preferably from 1 to 3%; and/or,
   HP5: from 1 to 15%, preferably from 2 to 10%, preferably from 3 to 8%; and/or,
   HP6: from 2 to 30%, preferably from 5 to 25%, preferably from 7 to 20%, preferably from 10 to 20%; and/or,
   HP7: from 5 to 45%, preferably from 10 to 40%, preferably from 15 to 35%, preferably from 20 to 30%; and/or,
   HP8: from 5 to 50%, preferably from 10 to 40%, preferably from 15 to 35%, preferably from 20 to 30%; and/or,
   HP9: from 3 to 40%, preferably from 5 to 35%, preferably from 10 to 30%, preferably from 11 to 25%, preferably from 15 to 25%; and/or,
   HP10: from 1 to 20%, preferably from 3 to 15%, preferably from 5 to 11%; and/or,
   HP11: from 0 to 10%, preferably from 1 to 5%; and/or,
   HP≥12: equal to or lower than 5%, preferably equal to or lower than 4%, preferably equal to or lower than 3%, preferably equal to or lower than 2%, preferably equal to or lower than 1%;
these percentages being expressed with respect to the sum of the signals obtained for each substitution for which the signal was higher than the one of the background; and/or,
(iii) a substitution pattern such that:
   the molar proportion of unsubstituted (no-OHP) moities is from 30 to 40%; and/or,
   the molar proportion of C2 substituted (2 OHP) moieties is from 25 to 35%; and/or,
   the molar proportion of C3 substituted (3 OHP) moieties from 6 to 7%; and/or,
   the molar proportion of C6 substituted (6 OHP) moieties is from 3 to 5%, preferably from 4 to 5%; and/or,
   the molar proportion of C2 and C3 substituted (2,3-di-OHP) moieties is from 10 to 15%, preferably from 11 to 14%; and/or,
   the molar proportion of C2 and C6 substituted (2,6-di-OHP) moieties is from 3 to 10%, preferably from 5 to 8%; and/or,
   the molar proportion of twice C3 substituted (3,3′-di-OHP) moieties is from 0.5 to 1.5%, preferably from 1.0 to 1.5%; and/or,
   the molar proportion of C2, C3 and C6 substituted (2,3,6-tri-OHP) moieties is from 1 to 5%, preferably from 2 to 4% preferably from 2 to 3%; and/or,
   the molar proportion of substitutions corresponding to mono-substitutions is from 60 to 70%; and/or,
   the molar proportion of substitutions corresponding to di-substitutions is from 25 to 35%, preferably from 30 to 35%; and/or,
   the molar proportion of substitutions corresponding to tri-substitutions is from 2 to 5%, preferably from 3 to 5%; and/or,
   the C2/C6 substitutions molar ratio is from 3.0 to 5.0, preferably from 3.0 to 4.5, preferably from 3.5 to 4.5, preferably from 3.5 to 4.0; and/or,
   the C2/C3 substitutions molar ratio is from 2.0 to 2.5.

Such hydroxypropyl-cyclodextrin is commercially available. For example, mention can be made of KLEPTOSE® HP (ROQUETTE FRERES).

In a second preferred embodiment, the hydroxypropyl-cyclodextrin according to the disclosure (e.g., HPBCD) has:
(i) a MS selected from 0.50 to 0.75, preferably from 0.55 to 0.70, preferably from 0.58 to 0.68; and/or,
(ii) the following substitution profile, as determined by electrospray ionization-Mass spectrometry (ESI-MS):
   HP≤1: equal to or lower than 5%, preferably equal to or lower than 4%, preferably equal to or lower than 3%, preferably equal to or lower than 2%, preferably equal to or lower than 1%; and/or,
   HP2: equal to or lower than 5%, preferably equal to or lower than 4%, preferably equal to or lower than 3%, preferably equal to or lower than 2%; and/or,
   HP3: from 0 to 15%, preferably from 1 to 10%, preferably from 1 to 8%, preferably from 2 to 6%; and/or,
   HP4: from 0 to 25%, preferably from 2 to 20%, preferably from 4 to 15%, preferably from 7 to 13%; and/or,
   HP5: from 1 to 40%, preferably from 5 to 35%, preferably from 10 to 30%, preferably from 15 to 25%; and/or,
   HP6: from 5 to 50%, preferably from 10 to 40%, preferably from 15 to 35%, preferably from 20 to 30%; and/or,
   HP7: from 5 to 50%, preferably from 10 to 40%, preferably from 15 to 35%, preferably from 20 to 30%; and/or,
   HP8: from 2 to 30%, preferably from 2 to 25%, preferably from 5 to 20%, preferably from 8 to 15%; and/or,
   HP9: from 1 to 10%, preferably from 1 to 8%, preferably from 2 to 5%; and/or,
   HP≥10: equal to or lower than 5%, preferably equal to or lower than 4%, preferably equal to or lower than 3%, preferably equal to or lower than 2%, preferably equal to or lower than 1%;
these percentages being expressed with respect to the sum of the signals obtained for each substitution for which the signal was higher than the one of the background and/or,
(iii) a substitution pattern such that:
   the molar proportion of unsubstituted (no-OHP) moities is from 40 to 50%; and/or,
   the molar proportion of C2 substituted (2 OHP) moieties is from 20 to 30%; and/or,
   the molar proportion of C3 substituted (3 OHP) moieties is from 7 to 8%; and/or,
   the molar proportion of C6 substituted (6 OHP) moieties is from 3 to 4%; and/or,
   the molar proportion of C2 and C3 substituted (2,3-di-OHP) moieties is from 5 to 12%, preferably from 6 to 10%; and/or,
   the molar proportion of C2 and C6 substituted (2,6-di-OHP) moieties is from 2 to 5%; and/or,
   the molar proportion of twice C3 substituted (3,3′-di-OHP) moieties is equal to or lower than 2%, preferably equal to or lower than 1.0%, for example from 0.4 to 1.0%, preferably from 0.5 to 0.9%; and/or,
   the molar proportion of C2, C3 and C6 substituted (2,3,6-tri-OHP) moieties is equal to or lower than 3%, preferably equal to or lower than 2%, preferably equal to or lower than 1.0, for example from 0.5 to 3%, preferably from 0.5 to 2.0%, preferably from 0.5 to 1.5%, preferably from 0.5 to 1.0% and/or
   the molar proportion of substitutions corresponding to mono-substitutions is from 70 to 80%; and/or,
   the molar proportion of substitutions corresponding to di-substitutions is from 20 to 30%; and/or,
   the molar proportion of substitutions corresponding to tri-substitutions is from 1 to 5%, preferably from 1 to 3%, preferably from 1.0 to 2.0%, preferably from 1.0 to 1.5%; and/or,
   the C2/C6 substitutions molar ratio is from 4.0 to 8.0, preferably from 4.5 to 7.5, preferably from 5.0 to 7.0, preferably from 5.0 to 6.5, preferably from 5.0 to 6.0; and/or,
   the C2/C3 substitutions molar ratio is from 2.0 to 2.5.

Such hydroxypropyl-cyclodextrin is commercially available. For example, mention can be made of KLEPTOSE® HPB (ROQUETTE FRERES).

In a third preferred embodiment, the hydroxypropyl-cyclodextrin according to the disclosure (e.g., HPBCD) has:
(i) a MS ranging from 0.50 to 0.80, preferably from 0.55 to 0.75, preferably from 0.58 to 0.71; and/or,
(ii) the following substitution profile, as determined by electrospray ionization-Mass spectrometry (ESI-MS):
   HP≤1: equal to or lower than 5%, preferably equal to or lower than 4%, preferably equal to or lower than 3%, preferably equal to or lower than 2%, preferably equal to or lower than 1%; and/or,
   HP2: equal to or lower than 5%, preferably equal to or lower than 4%, preferably equal to or lower than 3%, preferably equal to or lower than 2%; and/or,
   HP3: from 1 to 10%, preferably from 2 to 8%, preferably from 3 to 7%; and/or,
   HP4: from 2 to 25%, preferably from 5 to 20%, preferably from 6 to 17%, preferably from 8 to 15%; and/or,
   HP5: from 5 to 40%, preferably from 10 to 30%, preferably from 15 to 25%, preferably from 17 to 25%; and/or,
   HP6: from 5 to 45%, preferably from 10 to 40%, preferably from 15 to 35%, preferably from 20 to 30%, preferably from 24 to 28%; and/or,
   HP7: from 5 to 40%, preferably from 10 to 30%, preferably from 15 to 25%; and/or,
   HP8: from 2 to 20%, preferably from 5 to 20%, preferably from 5 to 15%; and/or,
   HP9: from 1 to 10%, preferably from 2 to 8%, preferably from 2 to 6%, and/or,
   HP≥10: equal to or lower than 5%, preferably equal to or lower than 4%, preferably equal to or lower than 3%, preferably equal to or lower than 2%, preferably equal to or lower than 1%;
these percentages being expressed with respect to the sum of the signals obtained for each substitution for which the signal was higher than the one of the background; and/or,
(iii) a substitution pattern such that:
   the molar proportion of unsubstituted (no-OHP) moities is from 40 to 50%; and/or,
   the molar proportion of C2 substituted (2 OHP) moieties is from 25 to 35%; and/or,
   the molar proportion of C3 substituted (3 OHP) moieties is from 7 to 8%; and/or,
   the molar proportion of C6 substituted (6 OHP) moieties is from 2 to 4%; and/or,
   the molar proportion of C2 and C3 substituted (2,3-di-OHP) moieties is from 7 to 12%, preferably from 9 to 10%; and/or,
   the molar proportion of C2 and C6 substituted (2,6-di-OHP) moieties is from 2 to 5%; and/or,
   the molar proportion of twice C3 substituted (3,3′-di-OHP) moieties is equal to or lower than 2%, preferably equal to or lower than 1.0%, for example from 0.4 to 1.0%, preferably from 0.5 to 0.9%; and/or,
   the molar proportion of C2, C3 and C6 substituted (2,3,6-tri-OHP) moieties is equal to or lower than 3%, preferably equal to or lower than 2%, preferably equal to or lower than 1.0, for example from 0.5 to 3%, preferably from 0.5 to 2.0%, preferably from 0.5 to 1.5%, preferably from 0.5 to 1.0% and/or
   the molar proportion of substitutions corresponding to mono-substitutions is from 70 to 80%; and/or,
   the molar proportion of substitutions corresponding to di-substitutions is from 20 to 30%; and/or,
   the molar proportion of substitutions corresponding to tri-substitutions is from 1 to 5%, preferably from 1 to 3%, preferably from 1.5 to 2.5%; and/or,
   the C2/C6 substitutions molar ratio is from 4.0 to 8.0, preferably from 4.5 to 7.5, preferably from 5.0 to 7.5; and/or,
   the C2/C3 substitutions molar ratio is from 2.0 to 2.5.

Such hydroxypropyl-cyclodextrin is commercially available. For example, mention can be made of KLEPTOSE® HPB-LB (ROQUETTE FRERES).

In another preferred embodiment, the substituted nonionic cyclodextrin compound according to the disclosure is methyl-cyclodextrin, preferably MBCD.

Preferably, the methyl-cyclodextrin according to the disclosure (e.g., MBCD), has an average molar substitution degree (MS) equal to or higher than 0.60. It is preferably equal to or lower than 0.90, preferably equal to or lower than 0.80, preferably equal to or lower than 0.70.

The MS of the methyl-cyclodextrin according to the disclosure can be determined by those skilled in the art by proton nuclear magnetic resonance (NMR), or by mass spectrometry (electrospray ionization mass spectrometry (ESI-MS) or matrix-assisted laser desorption/ionization mass spectrometry (MALDI-MS)). While these techniques are well known to those skilled in the art, the latter may for example refer to the methods described in the reference thesis by Roman Jacquet: “Cyclodextrines hydrophiles: caractérisation et étude de leurs propriétés enantiosélective et complexante. Utilisation de la chromatographie en phase liquide et de la spectrométrie de masse” [“Hydrophilic cyclodextrins: characterization and study of their enantioselective and complexing properties. Use of liquid chromatography and mass spectrometry”]. Thesis on the chemistry and physicochemistry of compounds of biological interest. University of Orléans, 2006. Especially available at: http://tel.archives-ouvertes.fr/docs/00/18/55/42/PDF/jacquet.pdf (consulted on Nov. 27, 2013), in particular Chapter 2, Part B (pages 59 to 83).

Preferably, the MS is determined by NMR, according to the following: measurements are taken at 25° C., and the calibration is carried out with the D₂O signal. Samples of MCD, and of unsubstituted cyclodextrin, are prepared at a concentration of 5 mg in 0.75 ml of D₂O. The solutions are evaporated to dryness under a nitrogen stream and then reconstituted in 0.75 ml of D₂O. This operation is repeated twice in order to ensure total exchange of the protons of the hydroxyl functions. The MS is calculated from the difference in integration between the spectrum of the unsubstituted cyclodextrin and that of the methyl cyclodextrin.

The NMR spectrum also makes it possible to calculate the substitution profile.

The methyl-cyclodextrin according to the disclosure (e.g., MBCD), may be substituted on the hydroxyl borne by the C2 carbon of the anhydroglucose units, or by the C3 and/or C6 carbons of the anhydroglucose units or by a combination of the C2, C3 and/or C6, preferably C2 and C6, carbons of the anydroglucose units.

Preferably, at least 50% of the methyl groups of the methyl-cyclodextrin according to the disclosure are located at the hydroxyl borne by the C2 carbon of the anhydroglucose unit, for example 50 to 80%, preferentially 60 to 80%, preferentially 65 to 80%, preferentially 70 to 80%, for example 75%.

In parallel, the other methyl groups are generally predominantly located at the hydroxyl borne by the C3 and/or C6 carbon of the anhydroglucose unit.

Preferably, the methyl-cyclodextrin according to the disclosure is methyl-β-cyclodextrin. In this case, the methyl-β-cyclodextrin preferentially has the following substitution profile:
0% to 5% of methyl-β-cyclodextrins comprise 2 methyl groups;
5% to 15% of methyl-β-cyclodextrins comprise 3 methyl groups;
20% to 25% of methyl-β-cyclodextrins comprise 4 methyl groups;
25% to 40% of methyl-β-cyclodextrins comprise 5 methyl groups;
15% to 25% of methyl-β-cyclodextrins comprise 6 methyl groups;
5% to 15% of methyl-β-cyclodextrins comprise 7 methyl groups;
0% to 5% of methyl-β-cyclodextrins comprise 8 methyl groups;
these percentages being molar percentages, and the total sum thereof being generally about 100%, although the composition may optionally contain traces of methyl cyclodextrins of different DS, and also traces of residual beta-cyclodextrin, i.e. unsubstituted cyclodextrin.

The substitution profile may be conventionally determined by those skilled in the art, for example by ESI-MS or MALDI-TOF-MS. Although these techniques are well known to those skilled in the art, those skilled in the art may for example refer to the methods described in the abovementioned thesis by Romain Jacquet in chapter 2, part B, points II.3 and II.2 (page 67 to 82) and in appendix II.

Preferably, the cyclodextrin compounds according to the disclosure for which a US monograph is available on May 1, 2022, do comply with the US monograph as in force on May 1, 2022.

Preferably, the cyclodextrins compound according to the disclosure for which a European monograph is available on May 1, 2022, do comply with the European monograph as in force on May 1, 2022.

Preferably, the cyclodextrins compound according to the disclosure for which a Chinese monograph is available on May 1, 2022, do comply with the Chinese monograph as in force on May 1, 2022.

Preferably, the total amount of nonionic or cationic cyclodextrin compound in the LNPs according to the disclosure is such that the molar ratio of total lipids constituting the LNPs to total nonionic and/or cationic cyclodextrin compound is from 1:1 to 500:1. It is preferably equal to or higher than 5:1, preferably equal to or higher than 10:1, preferably equal to or higher than 20:1, preferably equal to or higher than 30:1, preferably equal to or higher than 40:1, preferably equal to or higher than 50:1; preferably equal to or higher than 60:1, preferably equal to or higher than 70:1. It is preferably equal to or lower than 400:1, preferably equal to or lower than 300:1, preferably equal to or lower than 200:1, preferably equal to or lower than 150:1, preferably equal to or lower than 140:1, preferably equal to or lower than 130:1, preferably equal to or lower than 120:1, preferably equal to or lower than 110:1, preferably equal to or lower than 100:1, preferably equal to or lower than 90:1. It is for example from about 70:1 to about 85:1.

Preferably, the nonionic or cationic cyclodextrin compound is incorporated in the LNPs together with the nucleic acid. It is preferably incorporated in the LNPs via the aqueous phase. The nucleic acid and the cyclodextrin compound may be incorporated in the same particle or in different particles of the LNPs population. Preferably, the LNPs comprise at least one nanoparticle incorporating both a cyclodextrin compound and a nucleic acid. Without being bound by any theory, the inventors believe that the majority of the LNPs population has particles incorporating both the cyclodextrin compound and the nucleic acid.

### Nucleic Acid

The nucleic acid according to the disclosure are typically selected from those which can be used as therapeutics. They might be selected from ribonucleic acids (RNAs) or from deoxyribonucleic acids (DNAs) or from a mixture thereof.

The nucleic acids according to the disclosure might include modified and/or non-naturally occurring components such as one or more nucleobases, nucleosides, nucleotides, or linkers. For example, in RNAs, uridine might be replaced by pseudouridine, to reduce degradation by RNase. Modifications of the non-coding regulatory regions are also possible. Examples are 5′-cap modification and elongation of poly (A) tail.

Preferably, the DNAs and RNAs according to the disclosure, preferably mRNAs, have from 5 to 20 000 of bases. This number of bases depends on the type of DNA or RNA used, and is preferably equal to or lower than 15 000, preferably equal to or lower than 10 000, preferably equal to or lower than 5 000. It is in general higher than 5, even equal to or higher than 10. It is for example equal to or higher than 100, even equal to or higher than 200, even equal to or higher than 300, even equal to or higher than 400, even equal to or higher than 500, even equal to or higher than 600, even equal to or higher than 700, even equal to or higher than 800, even equal to or higher than 900.

Preferably, the nucleic acid according to the disclosure is selected from RNAs. The RNA according to the disclosure might be selected from messenger RNA (mRNA), small interfering RNA (siRNA), antisense RNA, asymmetrical interfering RNA (aiRNA), microRNAs (miRNA), dicer-substrate RNAs (dsRNA), small hairpin RNA (shRNA), transfer RNA (IRNA), long non-coding RNA (lncRNA), RNAi-inducing agent, RNAi agent, ribozyme, RNA that induce triple helix formation, aptamer, vector, shortmer, antagomir, or from any mixture thereof.

The RNA according to the disclosure may have a nucleotide sequence encoding a peptide or protein of interest. For example, an RNA may be an mRNA. Translation of an mRNA encoding a particular peptide or protein, for example, in vivo translation of an mRNA inside a mammalian cell, may produce the encoded peptide or protein.

Preferably, the RNA according to the disclosure comprises or consists in an mRNA and/or a hybrid thereof. The protein or peptide encoded by the mRNA may be of any size and may have any secondary structure or activity. In a preferred embodiment, a protein or peptide encoded by an mRNA has a therapeutic effect when expressed in a cell.

Generally, the shortest length of a nucleic acid can be the length of the polynucleotide sequence that is sufficient to encode for a dipeptide. Preferably, the length of the nucleic acid sequence is sufficient to encode for a peptide or protein having from 10 to 5000 amino acids and not more than 6,600 amino acids.

Preferably, the length of the nucleic acid sequence is sufficient to encode for a peptide or protein having at least 20 amino acids, preferably at least 30 amino acids, preferably at least 40 amino acids, preferably at least 50 amino acids. Preferably, the length of the nucleic acid sequence is sufficient to encode for a peptide or protein having no more than 5000 amino acids, preferably no more than 1000 amino acids, preferably no more than 500 amino acids, preferably no more than 400 amino acids, preferably no more than 300 amino acids.

The nucleic acid according to the disclosure may be also selected from DNAs. Examples of DNAs are catalytic DNAs or aptamers.

Preferably, the nitrogen: phosphate (N: P) molar ratio of the LNPs according to the disclosure is equal to or higher than 1:1, preferably equal to or higher than 2:1, preferably equal to or higher than 3:1, preferably equal to or higher than 4:1. It is preferably equal to or lower than 10:1, preferably equal to or lower than 9:1, preferably equal to or lower than 8:1, preferably equal to or lower than 7:1, preferably equal to or lower than 6:1. It is for example equal to about 4:1, or equal to about 6:1.

The “N: P ratio” is the molar ratio of cationic or cationizable nitrogen atoms in cationic/cationizable lipid to phosphate groups in the nucleic acid (e.g., RNA). This N: P ratio thus reflects the ratio of cationic/cationizable lipid to nucleic acid used. It is calculated from the amounts of cationic/cationizable lipid and nucleic acid used for LNPs preparation.

Preferably, the LNPs according to the disclosure have an encapsulation efficiency equal to or higher than 75%, preferably equal to or higher than 80%, preferably equal to or higher than 85%, preferably equal to or higher than 90%, preferably equal to or higher than 95%.

This encapsulation efficiency is the percentage by weight of nucleic acid that is encapsulated or otherwise associated with the LNPs after preparation of said LNPs. The encapsulation efficiency may be measured, for example, by comparing the amount of nucleic acid in a solution containing the LNPs before and after breaking up the LNPs with an organic solvents or detergent, for example with Tris-EDTA buffer (TE buffer). Fluorescence may be used to measure the amount of nucleic acid. The encapsulation efficiency might be determined according to the protocol described hereinafter in the Examples section.

Preferably, the LNPs according to the disclosure have improved potency, in particular as compared to same LNPs when the nonionic or cationic cyclodextrin compound is absent. Such potency might be determined for example by assaying the level of transfected nucleic acid in a cellular assay, for example using HEK cells or Hela cells. It can be determined for example by measuring the level of expression of a peptide or protein encoded by said nucleic acid. In the latter case, it can for example be determined by measuring the fluorescence produced by a fluorescent protein e.g., Green Fluorescent Protein (GFP) encoded by the nucleic acid. It can for example be determined according to the protocols given in the Examples section.

Preferably, the average diameter of the LNPs according to the disclosure is equal to or lower than 1000 nm, preferably equal to or lower than 800 nm, preferably equal to or lower than 600 nm, preferably equal to or lower than 400 nm, preferably equal to or lower than 200 nm, preferably equal to or lower than 150 nm, preferably equal to or lower than 140 nm, preferably equal to or lower than 130 nm, preferably equal to or lower than 120 nm, said average diameter being determined by dynamic light scattering (DLS). This average diameter is in general equal to or higher than nm, even equal to or higher than 20 nm, even equal to or higher than 40 nm, even equal to or higher than 50 nm.

To determine the average diameter by DLS, the person skilled in the art might follow the protocol as described in the Example section II.B.2.

Preferably, the polydispersity index (PDI) of the LNPs according to the disclosure is lower than 0.3, preferably equal to or lower than 0.2, preferably equal to or lower than 0.1, more preferably lower than 0.1, preferably equal to or lower than 0.09, preferably equal to or lower than 0.08. It is in general equal to or higher than 0.0001, even equal to or higher than 0.001. Such polydispersity index may be determined by the person skilled in the art by DLS, for example according to the protocol as described in the Example section II.B.2.

Preferably, the zeta potential of the LNPs according to the disclosure is equal to or higher than-10 mV and equal to or lower than +20 mV. It is still preferably equal to or higher than-5 mV. It is preferably equal to or lower than +15 mV, preferably equal to or lower than +10 mV, preferably equal to or lower than +5 mV. It is for example equal to about 0 mV.

The zeta potential might be determined by the person skilled in the art by DLS or potentiometry (e.g., potentiometric titrations).

The LNPs according to the disclosure might be non-specific or specific. In the latter case, the LNP classically includes a ligand able to interact with a receptor that is specific to one or more cell types.

The LNPs according to the disclosure might include other compounds than the ones described before, as long as it does not interfere with the desired properties of the LNPs, notably in terms of efficacy and safety. Such additional compounds might be selected from:
adjuvants, e.g., Glucopyranosyl Lipid Adjuvant (GLA), CpG oligodeoxynucleotides (e.g., Class A or B), poly (LC), aluminum hydroxide, and Pam3CSK4;
small hydrophobic molecules such as a vitamin (e.g., vitamin A or vitamin E);
permeability enhancer e.g., hydrogels and their synthetic polymers such as poly (acrylic acid)-based materials, environment responsive (especially thermo-sensitive) polymers, and innovative polysaccharide-based hydrogels.
carbohydrates other than the nonionic or cationic cyclodextrin compounds according to the disclosure, e.g., glucose;
polymers other than the nonionic or cationic cyclodextrin compounds according to the disclosure, e.g., glycogen and derivatives and analogs thereof;
surface altering agents, e.g., anionic proteins (e.g., bovine serum albumin), surfactants (e.g., cationic surfactants such as dimethyldioctadecyl-ammonium bromide), sugars or sugar derivatives, nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin b4, dornase alfa, neltenexine, and erdosteine), and DNases (e.g., rhDNase);
lipids other than the ones previously described;
dies;
or from any mixture thereof.

However, because it seems that such other compounds are not required in the LNPs according to the disclosure, the LNPs preferably include less than 10% by dry weight of such other ingredients, preferably less than 5%, preferably less than 2%, preferably less than 1%, preferably 0%. Still preferably, and because no additional compound seems to be required in order to obtain satisfying LNPs in the instant disclosure, the LNPs are preferably free of such additional compounds.

Preferably, the LNPs according to the disclosure consist in:
a cationic/cationizable lipid, preferably as described before;
a sterol, preferably as described before;
a PEG-lipid and/or a surfactant other than said PEG-lipid, preferably as described before;
a helper lipid (preferably a phospholipid), preferably as described before;
a nonionic or cationic cyclodextrin compound, preferably as described before;
a nucleic acid, preferably as described before;
less than 10% dry weight of other compounds, preferably as described before.

It is reminded that these ingredients might be composed of more than one substance. For example, the helper lipid might be a mixture of DOPE and DSPC.

The compositions of matter according to the disclosure may only consist in the LNPs according to the disclosure.

The composition of matter according to the disclosure may also comprise other ingredients, as long as it does not interfere with the desired properties, notably in terms of safety and efficacy.

These other ingredients can be selected from substances and/or solvents.

Examples of such other ingredients are:
solvents, preferably water;
cryoprotectants, for example glycerol, ethanol, sucrose;
a chelator, e.g., diethylenetriamine pentaacetic acid (DTPA), ethylenediaminetetraacetic acid (EDTA), iminodisuccinic acid, polyaspartic acid, ethylenediamine-N,iV-disuccinic acid (EDDS), methylglycinediacetic acid (MGDA), L-glutamic acid N,N-diacetic acid (GLDA);
an antioxidant, e.g., ascorbic acid, citric acid, malic acid, methionine, monothioglycerol, phosphoric acid, potassium metabisulfite, alpha-tocopherol;
a surfactant e.g., polysorbate;
a buffering agent, e.g., tromethamine, sodium acetate, dibasic sodium phosphate dihydrate, monobasic potassium phosphate;
a salt, for example potassium chloride, sodium chloride;
or any mixture thereof.

The nature and quantities of such other ingredients will depend on the intended use of the composition of matter according to the disclosure. Typically, the composition of matter according to the disclosure is intended to be administered to an organism, or is a composition useful for the preparation of a composition intended to de administered to an organism.

The compositions of matter according to the disclosure might be in the suspension form or in the dried state. In the latter case, it is preferably in the lyophilized form.

Preferably, said suspension is a suspension of LNPs in a phosphate buffer, preferably in a phosphate-buffered saline (PBS) solution.

In a preferred embodiment, the compositions of matter according to the disclosure are injectable compositions. In that case, the composition is typically a suspension of LNPs.

In a preferred embodiment, the compositions of matter according to the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C.,-130° C. or −150° C.). Typically, the composition according to the disclosure is a suspension or solid (e.g., via lyophilization) that is refrigerated for storage and/or shipment at, for example, about −20° C., −30° C., −40° C., −50° C., −60° C., −70° C., or −80° C.

### Process for Making LNPs, and LNPs Obtainable Thereof

The invention also relates to a process for making LNPs, comprising putting into contact lipids, a nucleic acid and a nonionic or cationic cyclodextrin compound, so as to obtain LNPs.

Preferably, the LNPs are as described before.

Preferably, the LNPs are prepared by rapid mixing. In this process, an aqueous phase comprising the nucleic acid is mixed with an organic phase comprising the lipid components. Solvents are typically selected so as to solubilize said nucleic acid and said lipid components.

Rapid mixing typically is performed by using high pressure, high shear mixing (homogenizer and microfluidizer), extrusion, microfluidic chip mixing, sonication, or freeze thaw. It is preferably performed by microfluidic chip mixing.

Typically, the solvent of the organic phase is selected from chloroform, ethanol, or a from a mixture thereof. It is preferably ethanol.

Preferably, the total amount of lipid components (concentration in the aqueous+organic phase) is equal to or higher than 0.1 mM, preferably equal to or higher than 0.5 mM, preferably equal to or higher than 1.0 mM, preferably equal to or higher than 2.0 mM, preferably equal to or higher than 3.0 mM, preferably equal to or higher than 4.0 mM, preferably equal to or higher than 5.0 mM. It is preferably equal to or lower than 100 mM, preferably equal to or lower than 50 mM, preferably equal to or lower than 40 mM, preferably equal to or lower than 30 mM, preferably equal to or lower than 20 mM, preferably equal to or lower than 15 mM, preferably equal to or lower than 10 mM, preferably equal to or lower than 9.0 mM, preferably equal to or lower than 8.0 mM, preferably equal to or lower than 7.0 mM. It is for example equal to about 5.0 to about 6.5 mM.

Preferably, the nonionic or cationic cyclodextrin compound is in the aqueous phase, i.e., with the nucleic acid. Preferably, the amount of nonionic or cationic cyclodextrin compound in the aqueous phase is equal to or higher than 0.01 mM, preferably equal to or higher than 0.05 mM. It is preferably equal to or lower than 15 mM, preferably equal to or lower than 10 mM, preferably equal to or lower than 5 mM, preferably equal to or lower than 4 mM, preferably equal to or lower than 3 mM, preferably equal to or lower than 2 mM, preferably equal to or lower than 1.0 mM, preferably equal to or lower than 0.5 mM. it is for example equal to about 0.1 mM.

Preferably, the volume ratio aqueous phase:organic phase (Aq: Org volume ratio) used in the process according to the disclosure is equal to or higher than 1:1, preferably equal to or higher than 2:1, equal to or higher than 3:1. It is preferably equal to or lower than 10:1, preferably equal to or lower than 9:1, preferably equal to or lower than 8:1, preferably equal to or lower than 7:1, preferably equal to or lower than 6:1, preferably equal to or lower than 5:1, preferably equal to or lower than 4:1. It is for example equal to about 3:1. Typically, in microfluidic mixing, this volume ratio is equal to the flow rate ratio. That is to say that the flows are selected such that the aqueous and organic phases start and end at the same time.

Preferably, the molar ratio of total lipids to total nonionic and/or cationic cyclodextrin compound used in the process is from 1:1 to 500:1. It is preferably equal to or higher than 5:1, preferably equal to or higher than 10:1, preferably equal to or higher than 20:1, preferably equal to or higher than 30:1, preferably equal to or higher than 40:1, preferably equal to or higher than 50:1; preferably equal to or higher than 60:1, preferably equal to or higher than 70:1. It is preferably equal to or lower than 400:1, preferably equal to or lower than 300:1, preferably equal to or lower than 200:1, preferably equal to or lower than 150:1, preferably equal to or lower than 140:1, preferably equal to or lower than 130:1, preferably equal to or lower than 120:1, preferably equal to or lower than 110:1, preferably equal to or lower than 100:1, preferably equal to or lower than 90:1. It is for example from about 70:1 to about 85:1.

Preferably, the LNP suspension obtained is buffer exchanged, preferably using a phosphate buffer, still preferably phosphate-buffered saline (PBS) solution.

The invention also relates to LNPs obtainable or obtained from the process for making LNPs according to the disclosure. The invention also relates to a composition of matter comprising said LNPs. Preferably, said composition of matter is as described before.

### Uses of the Composition of Matter Comprising LNPs

The invention also relates to the use of the composition of matter according to the disclosure, as a medicine, and/or as a vaccine, and/or in cell therapy, and/or in gene therapy, in particular in CRISPR-Cas9 based therapies, and/or in theranostic applications.

It can for example be used as a vaccine against pathogens like viruses, bacteria, fungi, prions, or parasites, or as a vaccine against cancer. It is preferably a vaccine against coronavirus, preferably a vaccine for coronavirus disease (COVID), preferably for COVID-19.

They are typically administered in the form of a suspension of LNPs.

The compositions of matter according to the disclosure are typically intended to be administered to an organism, preferably to a human or an animal, preferably to a mammal, preferably to a human. The composition of matter according to the disclosure might be administered by enteral route, parenteral route, preferably by parenteral route. It is preferably administered by intramuscular route, intravenous route, subcutaneous route, transcutaneous route, or pulmonary route, for instance by inhalation or intratracheal administration. The compositions of matter according to the disclosure might also be useful for the preparation of a composition intended to be administered to an organism.

The invention also relates to a method for treating an individual in need thereof, or for vaccinating an individual in need thereof, or for treating by cellular therapy an individual in need thereof, or for treating by gene therapy an individual in need thereof, or for the theranostic of an individual in need thereof, comprising administering a composition of matter according to the disclosure to said individual in need thereof.

### Use of a Cyclodextrin Compound for Improving LNPs

The invention also relates to a method, for improving the potency of LNPs and/or for increasing the quantity of protein produced from the nucleic acid of LNPs, and/or for increasing the release the nucleic acid of LNPs into the cell cytoplasm, and/or for increasing endosomal escape of the nucleic acid of LNPs, and/or for improving the stability of LNPs, comprising incorporating a nonionic or cationic cyclodextrin compound in said LNPs.

Preferably, the nonionic or cationic cyclodextrin compound is as described before. Preferably, the LNPs are as described before.

Preferably, the potency, or the quantity of protein produced, or the stability are improved as compared to same LNPs when the nonionic or cationic cyclodextrin compound is absent. Such improvement might be determined for example by assaying the level of transfected nucleic acid in a cellular assay, for example using HEK cells or Hela cells. It can be determined for example by measuring the level of expression of a peptide or protein encoded by said nucleic acid. In the latter case, in can for example be determined by measuring the fluorescence produced by a fluorescent protein, e.g., Green Fluorescent Protein (GFP) encoded by the nucleic acid. For evaluating the stability, this test can be performed at different time of storage for example at 4° C. or at 25° C. Still for evaluating the stability, the encapsulation efficiency and/or average diameter and/or polydispersity index can be performed at different time of storage for example at 4° C. or at 25° C.

Those tests can for example be performed according to the protocols given in the Examples section.

The invention also relates to a method for improving the potency of LNPs, and/or for increasing the quantity of protein produced from the nucleic acid of LNPs, and/or for increasing the release of the nucleic acid of LNPs into the cell cytoplasm, and/or for increasing endosomal escape of the nucleic acid of LNPs, and/or for increasing the stability of LNPs, comprising putting into contact lipids, a nucleic acid and a nonionic or cationic cyclodextrin compound, so as to obtain said LNPs.

Preferably, the LNPs are as described before. In particular, the nonionic or cationic cyclodextrin compound is preferably as described before.

Preferably, said methods are performed by carrying out the process for making LNPs according as described before.

In the instant disclosure, it is referred to amounts of materials constituting the LNPs (cyclodextrin compound, lipids etc.) It should be understood that these amounts refer to the amounts of starting materials used for making the LNPs. These amounts may differ from the amounts actually found the final LNPs, which cannot be measured as of today.

In the instant disclosure, when it is referred to “LNPs according to the disclosure”, it is referred to LNPs incorporating the nucleic acid and the cyclodextrin compound. That is to say that when it is referred for example to an amount of material with respect to the weight of LNPs, unless otherwise specified, this weight includes the weights of nucleic acid and cyclodextrin compound (used as a starting material for making LNPs, as explained above).

Other characteristics and advantages of the present invention will emerge clearly on reading the examples given hereinafter, which illustrate the invention without however limiting it.

### Examples

### I. Material

### A. Cyclodextrin Compounds

As cyclodextrin compounds the followings were used (Table 1)

TABLE 1

| Product name | Description | Reference |
|---|---|---|
| KLEPTOSE ® HPB | hydroxypropyl-β-cyclodextrin | HPBCD1 |
| (ROQUETTE) | with an MS of 0.58-0.68 (nonionic) | |
| KLEPTOSE ® HP | hydroxypropyl-β cyclodextrin | HPBCD2 |
| (ROQUETTE) | with an MS of 0.81-0.99 (nonionic) | |
| experimental | methyl-β-cyclodextrin compound as | MBCD1 |
| KLEPTOSE ® CRYSMEB | described in patent U.S. Pat. No. 11,098,135 B2 | |
| (ROQUETTE) | with an MS of 0.67 (nonionic) | |
| KLEPTOSE ® BETA- | β-cyclodextrin (nonionic) | BCD |
| CYCLODEXTRIN | | |
| (ROQUETTE) | | |
| CAPTISOL ® | sulfobutylether- β-cyclodextrin | SBEBCD1 |
| (LIGAND) | with an MS of 0.93 (anionic) | |

### B. Lipids

As lipids the followings were used (Table 2)

TABLE 2

| Product | Supplier | Lipid type |
|---|---|---|
| Cholesterol (C3045) | Sigma Aldrich | cholesterol compound |
| β-sitosterol (S1270) | Sigma Aldrich | cholesterol compound |
| SM-102 | Sinopeg | cationizable lipid |
| (CAS: 2089251-47-6) | | |
| DSPC (P1138) | Avanti Polar Lipids | helper lipid |
| DMG-PEG 2000 (880151P) | Avanti Polar Lipids | PEG-lipid |
| GenVoy-ILM ® | PNI/Polaris Science | training kit comprising |
| | | a lipid mix with a |
| | | concentration of |
| | | 6.25 mM. |

### C. Nucleic Acid

As nucleic acid, a mRNA coding for a green fluorescent protein (GFP) was used (CleanCap® EGPF mRNA (L-7601), TriLink Biotechnologies).

### D. Other Chemicals and Reagents

QuantiT™ RiboGreen® RNA (R11490) reagent and rRNA standards were purchased from Life Technologies. Glo™ Luciferase Assay (E2510) was purchased from Promega. EGFP (L-7601), were purchased from TriLink Biotechnologies. H/G ethanol were purchased from Tee hai Chem. Triton™ X-100 (T8787) was purchased from Sigma. All cell culture reagents were purchased from Thermofisher.

### II. Evaluation of the Use of Cyclodextrin Compounds in LNPs

A. LNPs with Lipid Composition 1 (GenVoy-ILM®)

### 1. Preparation of the LNPs

Different types of mRNA-LNPs were prepared, including or not cyclodextrins, referred to as follow (Table 3):

TABLE 3

| | Cyclodextrin | Amount of cyclodextrin used |
|---|---|---|
| mRNA-LNP reference | compound used | in the aqueous phase |
| LNP-Control | — | Not applicable |
| 0.1 mM LNP-MBCD1 | MBCD1 | 0.1 mM |
| 0.1 mM LNP-HPBCD1 | HPBCD1 | 0.1 mM |
| 5 mM LNP-HPBCD1 | HPBCD1 | 0.5 mM |

The mRNA-LNPs were prepared as follow:

Preparation of the organic phase: lipids were dissolved in ethanol. The composition of the lipids (“Lipid Composition 1”) was as presented in Table 4.

TABLE 4
Lipid Composition 1 (GenVoy-ILM ®)

| | % molar* |
|---|---|
| Cationic / cationizable lipid: unknown | 50% |
| Cholesterol compound: unknown | 37.5% |
| Helper lipid: DSPC | 10% |
| PEG-lipid: unknown | 2.5% |

*Percentages are expressed with respect to the total amount of lipids composing the LNPs.

Preparation of the aqueous phase: stock cyclodextrins were added at 0.1 mM or 5 mM to the formulation buffer and mixed prior to the addition of a fixed quantity of mRNA of 100 μg.

mRNA-LNPs assembly: the mRNA-LNPs were formulated using NanoAssemblr® Ignite (Precision NanoSystems Inc.), at a constant flow rate. The optimized process parameters used were as presented in Table 5.

TABLE 5

| N:P ratio | 4:1 | |
|---|---|---|
| Flow rate | 12 | mL/min |
| Aq:Org volume ratio | 3:1 | |
| Total amount of lipid components | 6.23 | mM |
| (concentration in the aqueous + organic phase) | | |
| Total formulation volume | 2 | mL |

The mRNA-LNPs suspensions were then buffer exchanged with phosphate-buffered saline (PBS, pH 7.4; 1 mL LNP+40 mL PBS) and concentrated using 30K MWCO Amicon Ultra centrifugal filters (3,000 rpm, 30-60 min per 15 mL).

### 2. LNPs Evaluation

The average diameter, polydispersity index (PDI), encapsulation efficiency, and transfection efficiency of the mRNA-LNPs thus prepared were then evaluated.

Hydrodynamic diameter (average diameter) and the PDI of the LNPs were determined at room temperature by Dynamic/Static Light Scattering using the Zetasizer Nano-ZS by diluting the samples 10× with software parameter setting presented below as recommended by Precision NanoSystems Inc.

To determine mRNA encapsulation efficiency of the LNPs, the latter (or PBS, blank) were diluted in TE buffer. Quant-iT™ RiboGreen® reagent was added, and fluorescence signal was quantified using Tecan Multimode Plate Reader. The encapsulation efficiency is the percentage by weight of mRNA encapsulated within the LNP compared to the total mRNA contained in the sample tested.

Transfection efficiency was assayed by measuring the amount of protein produced by transfected cells, according to the following protocol:
cell culture: HEK293 cells (50 ul of 0.65M/ml per well of 96 well) were grown in appropriate growth media supplemented with 10% fetal bovine serum. All cultures were grown in 37° C. incubators supplemented with 5% CO₂ and were cultured according to suppliers' instructions.
transfection: After RiboGreen® assay, the mRNA-LNPs suspensions were added (in an amount such that a fixed 50 ng of mRNA or a fixed 70 g of mRNA were used) into respective culture media and topped up to 50 μl each well and added into three wells for each sample for technical accuracy. Upon the addition of LNPs samples, HEK293 cells were trypsinized and added to each well of a 96-well plate at a volume of 50 μl at a cell density of 0.65 million cells/ml. After 2 hours, an additional 100 μl culture media was added into each well. Tecan measurement was performed 24 hours after transfection. This allowed for the quantification of protein produced, thus reflecting the transfection efficiency.

The encapsulation efficiency, average diameter and PDI were comparable for all LNPs. In particular, the PDI was consistently lower than 0.2.

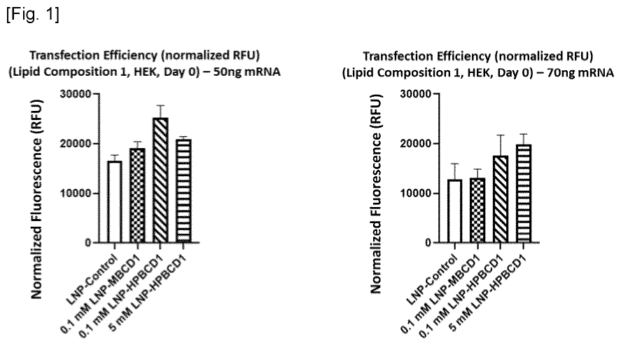

Results of transfection are presented in FIG. 1. The transfection efficiency was higher in the LNPs including a cyclodextrin compound, as compared to the control, not including any cyclodextrin compound. These results demonstrate the higher potency of the LNPs according to the invention. The use of 0.1 mM cyclodextrin seems to be preferred over the use of 5 mM cyclodextrin.

The inventors then evaluated the stability of the mRNA-LNPs, by measuring the average diameter, PDI, encapsulation efficiency, and transfection efficiency of the mRNA-LNPs stored at 4° C., at different timepoints. For these experiments, a cyclodextrin amount of 0.1 mM was used in the aqueous phase.

The encapsulation efficiency, average diameter and PDI were acceptable and steady over the time for all LNPs. These results show that the introduction of a cyclodextrin compound into the LNPs does not seem to impair their stability.

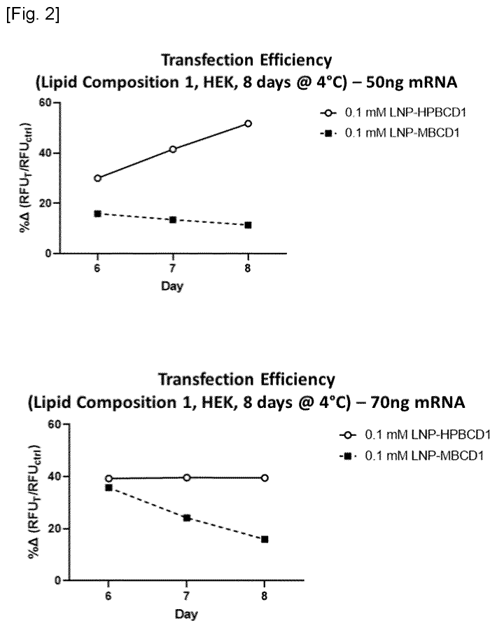

The results of transfection efficiency are presented in FIG. 2, in which the transfection efficiency was plotted against the LNP-Control, not including any cyclodextrin compound. The LNPs according to the disclosure comprising a nonionic cyclodextrin all exhibited a higher transfection efficiency as compared to the control. That is to say that the LNPs according to the disclosure comprising a nonionic cyclodextrin had improved stability. The mRNA-LNPs “LPN-MBCD1” and “LPN-HPBCD1” according to the disclosure showed higher potency as compared to the control that did not contain any cyclodextrin compound. This higher potency of the LNPs according to the disclosure was maintained over the time. After 8 days of storage at 4° C., the LNPs according to the disclosure all exhibited a higher transfection efficiency, as compared to the control not including any cyclodextrin.

The higher potency and stability of the LNPs according to the disclosure may offer the possibility to decrease the doses of mRNA-LNPs to be administered.

The inventors then decided to check whether the use of cyclodextrins could be applicable to other types of lipid systems, and whether other cyclodextrin compounds could be used.

B. LNPs with Lipid Composition 2

In this section, the inventors tested another lipid system (used in the COVID-19 vaccine marketed by MODERNA), and other cyclodextrin compounds. The LNPs stability was also further evaluated by measuring the encapsulation efficiency and particle size of the LNPs over the time.

### 1. Preparation of the LNPs

Different types of mRNA-LNPs were prepared, including or not cyclodextrins, referred to as follow (Table 6):

TABLE 6

| mRNA-LNP reference | Cyclodextrin compound used |
|---|---|
| LNP-Control | — |
| LNP-BCD | BCD |
| LNP-SBEBCD1 | SBEBCD1 |
| LNP-MBCD1 | MBCD1 |
| LNP-HPBCD1 | HPBCD1 |
| LNP-HPBCD2 | HPBCD2 |

The mRNA-LNPs were prepared as described in section A.1., except that the lipid composition was as presented in Table 7 (“Lipid Composition 2”).

TABLE 7
Lipid Composition 2

| | % molar* |
|---|---|
| Cationizable lipid: SM-102 | 50% |
| Cholesterol compound: cholesterol | 38.5% |
| Helper lipid: DSPC | 10% |
| PEG-lipid: DMG-PEG 2000 | 1.5% |

*Percentages are expressed with respect to the total amount of lipids composing the LNPs.

The amount of cyclodextrin compound used in the aqueous phase was of 0.1 mM

The optimized process parameters used were the following (Table 8).

TABLE 8

| N:P ratio | 5.67:1 | |
|---|---|---|
| Flow rate | 9 | mL/min |
| Aq:Org volume ratio | 3:1 | |
| Total amount of lipid components | 5.5 | mM |
| (concentration in the aqueous + organic phase) | | |
| Total formulation volume | 2.4 | mL |

### 2. LNPs Evaluation

The encapsulation efficiency, average diameter, PDI and transfection efficiency were measured at Day 0 (TO) and also upon storage at 4° C. or 25° C., for about 2-3 weeks. The encapsulation efficiency, average diameter and PDI were measured as in section A.2.

Transfection efficiency of the mRNA-LNPs was evaluated in two different cell types, according to the following protocol:
Cell culture: HEK293 cells (50 μl of 0.65M/ml per well of 96 well) and Hela cells (50 μl of 0.45M/ml per well of 96 well) were grown and seeded as described in section A.2.
Transfection: After RiboGreen® assay, the mRNA-LNPs suspensions were added (in an amount such that a fixed 50 ng of mRNA was used) into respective culture media and topped up to 50 μl each well and added into three wells for each sample for technical accuracy. Upon the addition of LNP samples, HEK293 or Hela cells were trypsinized and added to each well of a 96-well plate at a volume of 50 μl at a cell density of 0.65 million cells/ml for HEK293 cells or of 0.45 million cells/ml for Hela cells. After 2 hours, an additional 100 μl culture media were added into each well. Tecan measurement was performed 24 hours after transfection

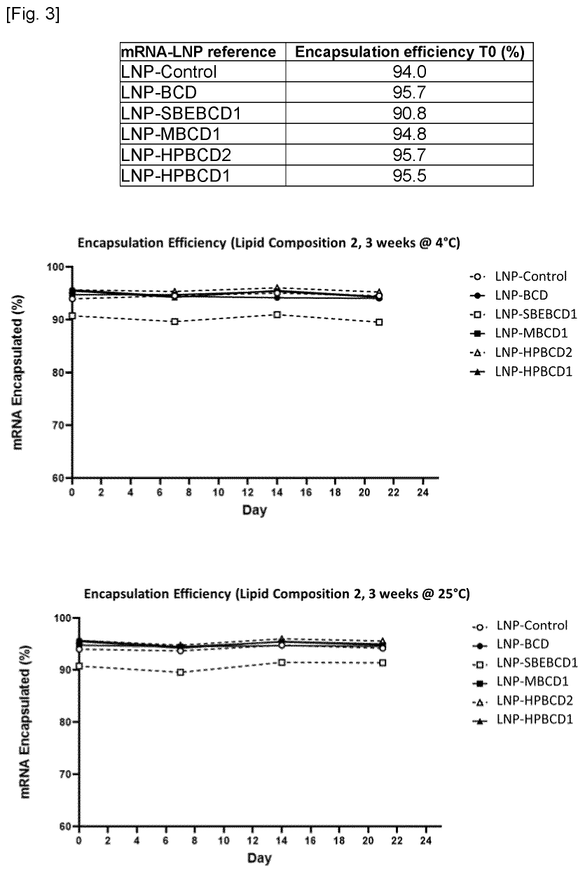

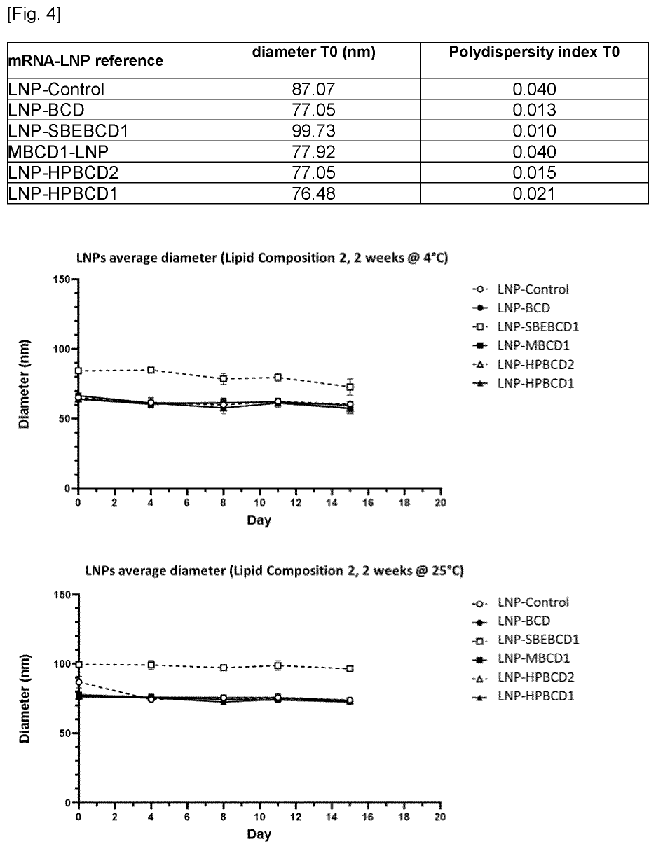

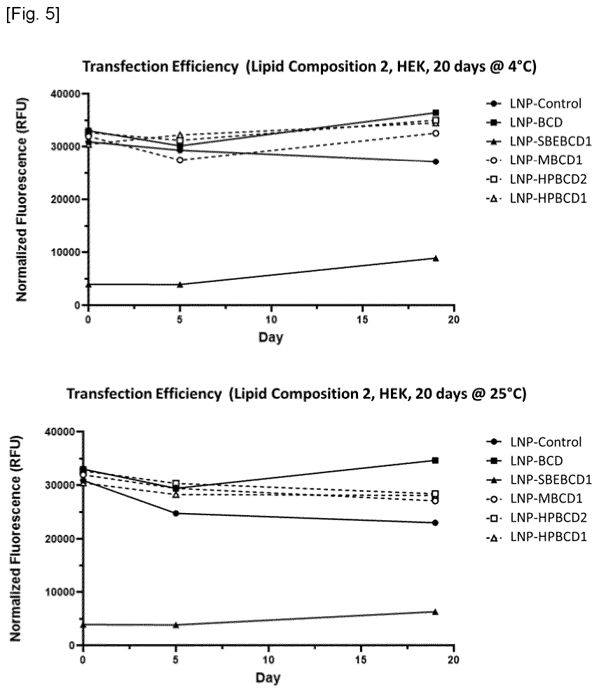

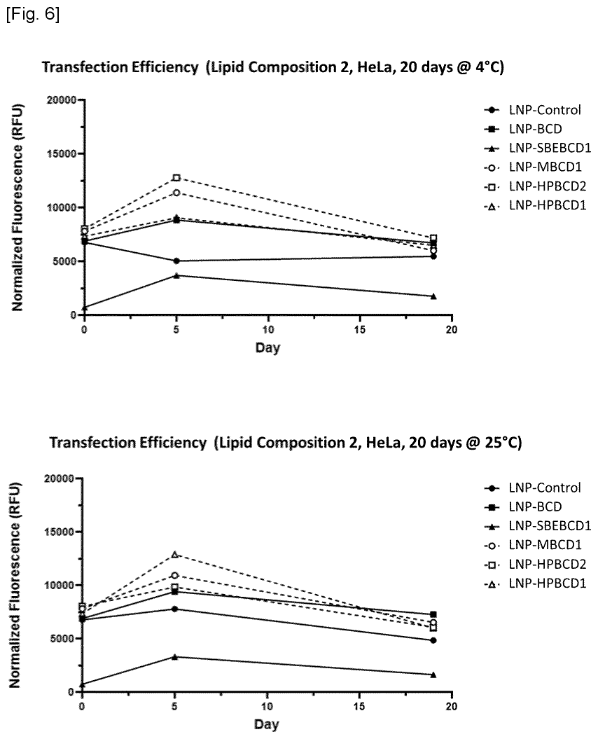

Results of encapsulation efficiency and diameter are presented FIGS. 3 and 4. Results of transfection efficiency are presented in FIGS. 5 and 6.

The encapsulation efficiency, average diameter and PDI were steady over the time for all LNPs. For all LNPs, the PDI (not showed in the graphs) was always lower than 0.2, typically from 0.01 to 0.13. These results show that the introduction of a cyclodextrin compound into the LNPs does not seem to impair their stability.

The encapsulation efficiency of the LNPs according to the disclosure was higher than 90%, which is very good. It can be noted that the LNP-SBEBCD1 including an anionic cyclodextrin compounds exhibited a slightly lower encapsulation efficiency and slightly higher diameter.

The mRNA-LNPs “LNP-BCD”, “LPN-MBCD1”, “LPN-HPBCD1” and “LPN-HPBCD2” according to the disclosure showed higher potency as compared to the control “LNP-Control” that did not contain any cyclodextrin compound. On the contrary, it was found that the use of the anionic cyclodextrin compound “SBEBCD1” was detrimental to the LNPs' potency: the transfection efficiency of comparative “LNP-SBEBCD1” was lower than the control. This higher potency of the LNPs according to the disclosure was maintained over the time. After about 20 days of storage at 4° C. or 25° C., the LNPs according to the disclosure all exhibited a higher transfection efficiency, as compared to the control not including any cyclodextrin.

C. LNPs with Lipid Composition 2 Using-Sitosterol Instead of Cholesterol

In this section, LNPs comprising another type of sterol were evaluated.

### 1. Preparation of the LNPs

Different types of mRNA-LNPs were prepared, including or not cyclodextrins, referred to as follow (Table 9):

TABLE 9

| mRNA-LNP reference | Cyclodextrin compound used | Sterol used |
|---|---|---|
| LNP-Control | — | cholesterol |
| LNP-β-Control | — | β-sitosterol |
| LNP-β-HPBCD1 | HPBCD1 | β-sitosterol |
| LNP-β-HPBCD2 | HPBCD2 | β-sitosterol |
| LNP-β-MBCD1 | MBCD1 | β-sitosterol |
| LNP-β-BCD | BCD | β-sitosterol |
| LNP-β-SBEBCD1 | SBEBCD1 | β-sitosterol |

LNPs were prepared as described in section B.1, except that cholesterol was substituted by β-sitosterol in Lipid composition 2 (“Lipid Composition 3”), except for LNP-Control.

### 2. LNPs Evaluation

The encapsulation efficiency, average diameter, PDI, and transfection efficiency were measured at Day 0 (TO) and also upon storage at 4° C. or 25° C., for 7 days. The encapsulation efficiency, average diameter and PDI were measured as in section A.2. As controls, LNPs with cholesterol or β-sitosterol without cyclodextrins were used.

Transfection efficiency of the mRNA-LNPs was evaluated on Hela cells, according to the protocol described in section B.2.

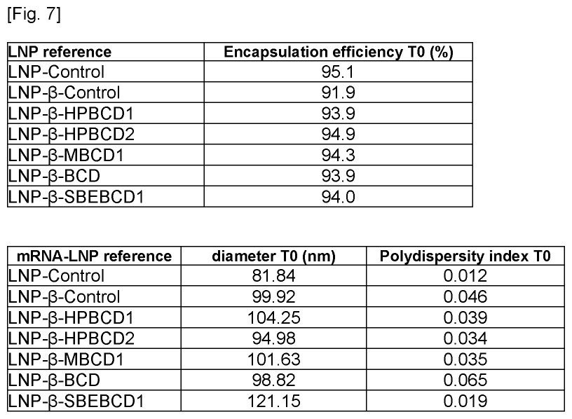

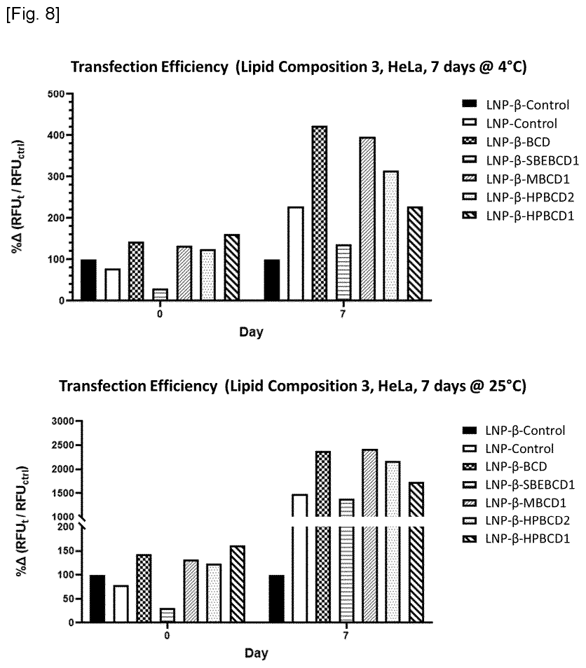

Results of encapsulation efficiency, average diameter and PDI at TO are presented in FIG. 7. Results of transfection efficiency are presented in FIG. 8.

The encapsulation efficiency, average diameter, and PDI were steady over the time for all LNPs (data not shown in the Figures). In particular, the PDI was always lower than 0.2. These results show that the introduction of a cyclodextrin compound according to the disclosure into the LNPs does not seem to impair their stability. The encapsulation efficiency of all LNPs was higher than 90%, which is very good. It can be noted that the diameter of LNPs comprising β-sitosterol is higher, as compared to the LNPs comprising cholesterol and even higher for comparative LNP-SBEBCD1 including an anionic cyclodextrin compound.

By comparing the control using cholesterol to the control using β-sitosterol, it can be observed that the substitution of cholesterol by β-sitosterol impairs the transfection efficiency of the LNPs after 7 days in both conditions (4° C. and 25° C.). However, the addition of the cyclodextrin compounds according to the disclosure

“MBCD1”, “HPBCD1”, “HPBCD2” and “BCD”, as well as substituting cholesterol with β-sitosterol, greatly improved the LNPs stability. The transfection efficiency of the LNPs was even greater than the control using cholesterol. On the contrary, the use of the comparative anionic cyclodextrin “SBEBCD1” did not allow to improve the stability of the LNPs as compared to the control using cholesterol as a helper lipid.

The data presented in this section II. altogether show the broad range of application of the technical solution provided herein. It can be applied to different cell types and different lipid systems. These data also show that different types cyclodextrin compounds can be used.

### III. Comparative Example: Use of Cyclodextrin Compounds in the LNPs Final Buffer (i.e., not in the LNP Itself)

mRNA-LNPs free of cyclodextrins were prepared, including or not cyclodextrins in the buffer, referred to as follow (Table 10):

TABLE 10

| mRNA-LNP reference | Cyclodextrin compound used in the buffer |
|---|---|
| LNP-Control | — |
| LNP + 0.1 mM MBCD1 | MBCD1 |
| LNP + 0.1 mM HPBCD1 | HPBCD1 |
| LNP + 0.1 mM BCD | BCD |
| LNP + 0.1 mM SBEBCD1 | SBEBCD |

Cyclodextrin-free mRNA-LNPs were prepared as in section B.1., and buffer exchanged using 30K MWCO Amicon Ultra centrifugal filters by replacing with 0.1 mM of respective cyclodextrin compounds diluted in PBS, or PBS only (control).

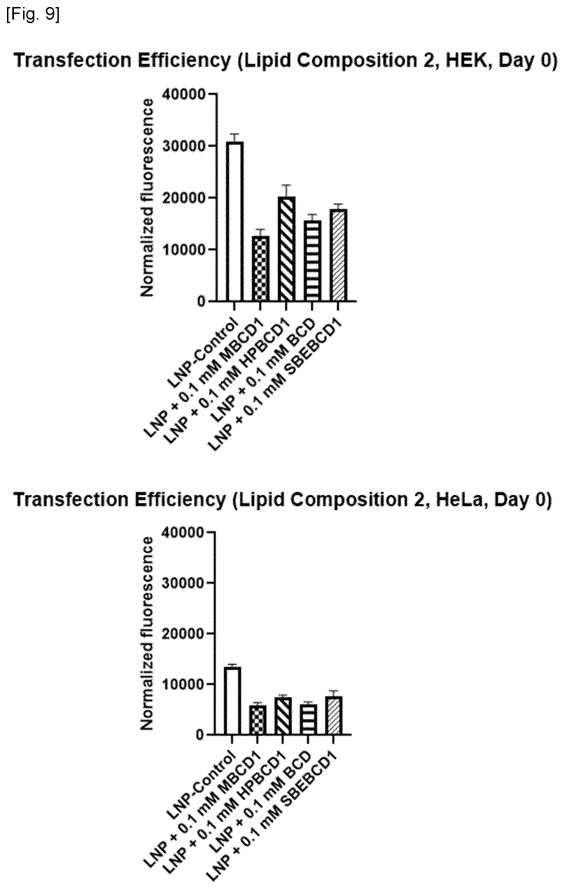

Results are presented in FIG. 9.

These results show that when the cyclodextrin compound is used in the buffer instead of being used in the structure of the LNPs, the potency of the LNPs is negatively impacted.

1. A composition of matter comprising lipid nanoparticles (LNPs), said lipid nanoparticles comprising lipids, a nucleic acid, and a nonionic or cationic cyclodextrin compound, said nucleic acid and cyclodextrin compound being incorporated into the nanoparticle structure comprising said lipids.

2. (canceled)

3. The composition of matter of claim 1, wherein said cyclodextrin compound is selected from a native cyclodextrin, a substituted cyclodextrin, or from a mixture thereof.

4. (canceled)

5. (canceled)

6. The composition of matter of claim 3, wherein said cyclodextrin compound is selected from a native cyclodextrin, a hydroxypropyl-cyclodextrin, a methyl-cyclodextrin, or from a mixture thereof.

7. The composition of matter of claim 1, wherein said lipids comprise a cationic or a cationizable lipid.

8. (canceled)

9. The composition of matter of claim 1, wherein said lipids comprise a polyethylene glycol (PEG)-lipid and/or a surfactant other than said (PEG)-lipid.

10. The composition of matter of claim 1, wherein said lipids comprise a helper lipid.

11. The composition of matter of claim 1, wherein said lipids comprise (a) a cationic or a cationizable lipid, (b) a sterol, (c) a (PEG)-lipid and/or a surfactant other than said (PEG)-lipid, and (d) a helper lipid.

12. (canceled)

13. (canceled)

14. (canceled)

15. (canceled)

16. A process for making LNPs, comprising putting into contact lipids, a nucleic acid and a nonionic or cationic cyclodextrin compound, so as to obtain said LNPs.

17. LNPs obtainable from the process of claim 16.

18. A composition of matter comprising the LNPs of claim 17.

19. (canceled)

20. (canceled)

21. (canceled)

22. (canceled)

23. (canceled)

24. (canceled)

25. A method for improving the potency of LNPs, comprising incorporating a nonionic or cationic cyclodextrin compound in said LNPs.

26. A method for increasing the quantity of protein produced from the nucleic acid of LNPs, comprising incorporating a nonionic or cationic cyclodextrin compound in said LNPs.

27. (canceled)

28. (canceled)

29. A method for increasing the stability of LNPs, comprising incorporating a nonionic or cationic cyclodextrin compound in said LNPs.

30. The composition of matter claim 1, wherein said lipids comprise a sterol.

31. The composition of matter of claim 30, wherein said sterol is selected from cholesterol, C-24 alkyl steroids, or any mixture thereof.

32. A method for treating an individual in need thereof, comprising administering the composition of matter of claim 1 to said individual in need thereof.

33. A method for vaccinating an individual in need thereof, comprising administering the composition of matter of claim 1 to said individual in need thereof.

34. A method for treating by cellular therapy an individual in need thereof, comprising administering the composition of matter of claim 1 to said individual in need thereof.

35. A method for treating by gene therapy an individual in need thereof, comprising administering the composition of matter of claim 1 to said individual in need thereof.

36. A method for the theranostic of an individual in need thereof, comprising administering the composition of matter of claim 1 to said individual in need thereof.
